(19) United States
(12) Patent Application Publication
(10) Pub. No.: US 2026/0364040 A1
(43) Pub. Date: Nov. 19, 2026

(54) MONO- AND BIS-NITROSYLATED PROPANEDIOLS FOR USE IN THE TREATMENT OF A THROMBOEMBOLIC DISEASE

(71) Applicant: ATTGENO AB, Solna (SE)

(72) Inventors: Per Håkan Agvald, Stockholm (SE); Leif Christofer Adding, Åkersberga (SE); Kristofer Bo Ingemar Nilsson, Örebro (SE); Anna Stene Hurtsén, Örebro (SE); Magnus Grenegård, Linköping (SE)

(21) Appl. No.: 19/478,479

(22) PCT Filed: Apr. 29, 2024
(86) PCT No.: PCT/EP2024/061806

§ 371 Date: Oct. 24, 2025

(30) Foreign Application Priority Data
Apr. 27, 2023 (GB) 2306258.1

(51) Int. Cl. A61K 31/21; A61P 7/02; A61P 9/08; A61P 9/12

(52) U.S. Cl. CPC A61K 31/21; A61P 7/02; A61P 9/08; A61P 9/12

(57) ABSTRACT

Described are methods of treating a condition, wherein such treatment includes administering certain mono- and/or bis nitrosylated propanediols, including compositions and formulations thereof, and wherein said administration is to a patient in need thereof via intra-arterial infusion in a dose of from about 0.01 to 3000 nmol kg-1 min-1. Also described is the use of mono- and/or bis nitrosylated propanediols for treating thromboembolic disease.

### FIELD OF INVENTION

The present invention relates to methods of treating a condition, wherein such treatment comprises administering certain mono- and/or bis-nitrosylated propanediols, including compositions and formulations thereof, and wherein said administration is to a patient in need thereof via intra-arterial infusion in a dose of from about 0.01 to 3000 nmol kg⁻¹ min⁻¹.

The present invention also relates to the use of mono- and/or bis-nitrosylated propanediols for treating thromboembolic disease.

### BACKGROUND OF THE INVENTION

The listing or discussion of an apparently prior-published document in this specification should not necessarily be taken as an acknowledgment that the document is part of the state of the art or common general knowledge.

Organ ischemia, dysfunction and failure after major surgery and in critical illness are devastating complications causing significant comorbidity and mortality. A specific medical therapy targeted to treat the ischemia (e.g. increasing blood flow by vasodilation, enhanced collateral blood flow, avoiding local formation of microthrombi/emboli by platelet inhibition, reducing reperfusion injury by inhibiting oxygen radical production) is lacking.

Specifically, uncontrolled activation of platelets is a key pathological event in acute thromboembolism, vessel occlusion and ischemic tissue damage, including mechanisms of thromboinflammation and immune-thrombosis. Pulmonary embolism represents a disease condition in which the interplay between several vital components are represented: cardiovascular and respiratory systems, inflammation and coagulation.

One common and serious form of organ ischemia is those conditions caused by acute arterial occlusion, which is a medical emergency where the damage caused depends on how long the affected organ will tolerate ischemia, which is a few minutes in the brain and roughly 4-6 hours in a limb. Symptoms of arterial occlusion include pain and loss of function. The longer these symptoms are present, the less likely it is to salvage the organ.

Dissolving or removing a blood clot to revascularize tissue is crucial.

Nitric oxide (NO) is a molecule of importance in several biological systems. It has become generally recognised that endogenous NO is of critical importance as a mediator of vasodilation in blood vessels.

With age, endogenous NO production ceases in different organs leads to increased risk of malfunction and disease. Impaired NO production is associated with increased risk for cardiovascular diseases including myocardial infarction, stroke and peripheral ischemia also called limb ischemia.

NO-donors have been in use since the mid-19th century to treat heart disease, the most well-known being nitroglycerin. However, there are problems with currently available NO donors including tolerance development and the risk of side effects attributed to the release of NO throughout the entire circulatory system. Thus, with the most used existing organic nitrate NO-donors increasingly high doses are required to maintain effect. For example, in the treatment of tissue at risk of damage due to ischemia, this usefulness of current available NO donors is restricted since when increasing the dose of existing NO-donors the vasodilatory effect elicited could cause dangerous drop in blood pressure when vessels in the entire vascular system is widened by the NO released.

Since the discovery that nitroglycerin and related compounds operate through the release of NO, considerable resources have been invested in attempts to develop new and better NO donors. However, it has now been more than 30 years since the ground-breaking discoveries relating to NO were made, and slow progress has been made in developing improved NO donors with higher specificity and fewer side effects.

To explain further, the most commonly used nitrates in a clinical setting, such as nitroglycerin, are presently used to treat the symptoms of angina (chest pain). Organic nitrates work by relaxing blood vessels and increasing the supply of blood and oxygen to the heart while reducing its workload. Examples of presently available organic nitrate drugs include:
a) Nitroglycerin (glyceryl trinitrate) (1,2,3-propanetriol-nitrate), which is today mostly taken sublingually to curb an acute attack of angina. However, strong headaches and dizziness due to the rapid and general vasodilatory effect are frequently encountered side-effects. Nitroglycerin infusion concentrates are also available and are diluted in isotonic glucose or physiological saline for intravenous infusion. Tolerance development (i.e. diminished efficacy with repeated or continuous dosage) is a clinical problem with Nitroglycerine (and other organic nitrates) treatment.
b) Isosorbide mononitrate (1,4:3,6-dianhydro-D-glucitol-5-nitrate), which is taken as prophylactic against angina pectoris. Tolerance development is a problem in long-term treatment regimens. Frequent side-effects include headache and dizziness, as encountered with nitroglycerin.
c) Isosorbide dinitrate (1,4:3,6-dianhydro-D-glucitol-2,5-nitrate), which is taken both acutely and prophylactically against angina pectoris and cardiac insufficiency.
d) Pentaerythrityl nitrates, a group of organic nitrates, which are known to exert long-term antioxidant and anti-atherogenic effects by currently unidentified mechanisms. Pentaerythrityl tetranitrate has been investigated in the context of nitrate tolerance, an unwanted development in nitrate therapy, and experimentally tested in pulmonary hypertension.

A number of these organic nitrate compounds, as well as other nitrate and nitrite compounds, have been tested in vivo and found to generate NO. For example, glyceryl trinitrate, ethyl nitrite, isobutyl nitrate, isobutyl nitrite, isoamyl nitrite and butyl nitrite have been tested in a rabbit model and were found to give a significant correlation between the in vivo generation of NO and effects on blood pressure (Cederqvist et al., Biochem. Pharmacol., 1994, 47, 1047-53).

With the growing knowledge regarding the importance of nitric oxide also the importance of dietary composition has been recognised since it could influence the availability of NO in the arginine-nitric oxide system and its role in host defence has been discovered (Larsen et al., N. Eng. J. Med, 2006, 355, 2792-3). Hence, L-arginine, and esters thereof, such as the ethyl-, methyl- and butyl-L-arginine have been used to increase the endogenous production of NO.

WO 2006/031191 describes compositions and methods for use in the therapeutic delivery of gaseous nitric oxide. Such compositions for the delivery of the gaseous NO comprise a compound capable of forming a reversible bond or association to NO, such as alcohols, carbohydrates and proteins.

WO 2007/106034 describes methods for producing organic nitrites from a compound which is a mono/polyhydric alcohol, or an aldehyde- or ketone-derivate thereof. The methods involve the de-aeration of an aqueous solution of said compound, followed by purging with gaseous nitric oxide (NO).

Nilsson, K. F. et al., Biochem Pharmacol., 82(3), 248-259 (2011) discusses the formation and identification of new bioactive organic nitrites.

WO 2020/109420 describes processes for making mono- and/or bis-nitrosylated propanediols along with the resulting compositions prepared. This document explains that such compounds are capable of treating conditions where NO has a beneficial effect.

WO 2021/239906 describes how these compounds have surprisingly been shown to be effective in treating microbial infections.

The inventors have previously shown that a mixture of mono- and/or bis-nitrosylated propanediols (PDNO), when infused to the lung is liberating NO with an ultrashort half-life making causing a predominant effect in the lung blood circulation. However, the mechanism behind this rapid decomposition of PDNO in the lung blood circulation is not known and since it surprisingly was found by the researchers that PDNO could reach the lung blood circulation and exerts its action even when given by other administrative routes, e.g., transdermal one would believe that the breakdown of PDNO in other parts of the body should generally be less effective and quick as compared to the lungs (WO 2021/239892).

Also, the knowledge behind the mechanisms for NO release from NO-donors is still, despite over 50 years of research on the mechanism of action for the liberation of NO from NO-donors (including organic nitrites) is still not clear. The majority of mechanistic studies has been performed in vitro and to bridge this to understanding of what actually happens in the extremely complex in vivo surrounding is almost impossible.

Beside the involvement of different enzymatic and non-enzymatic passages to metabolise NO-donors, the effectiveness of these could be influenced by the local environment in the organs where the NO liberation from the molecules occur such as local pH, the levels of oxygen and carbon dioxide, state of inflammation in the tissue etc. Also, the bioavailability of the NO released by a specific NO-donor might vary due to various factors such as the concentration of oxygen radicals in the tissues, which in turn could be influenced by the metabolism of the NO donors per se, i.e. the release of NO from nitroglycerin has been shown to increase the levels of oxygen radicals which in turn might contribute to tissue damage.

Altogether the complexity of the bioconversion of organic nitrates and NO-biology per se makes it almost impossible to foresee which action a given dose of an NO-donor would have in a specific organ especially if this is affected by pathological processes such as ischemia or infection.

A large amount of research has been focused on intravenous administration of NO donors.

Low doses by intravenous infusion of PDNO have been shown to exert vasodilation mainly in the pulmonary circulation (Nilsson K F, Lundgren M, Agvald P, Adding L C, Linnarsson D, Gustafsson L E. Formation of new bioactive organic nitrites and their identification with gas chromatography-mass spectrometry and liquid chromatography coupled to nitrite reduction. Biochem Pharmacol. 2011; 82(3):248-59; and Nilsson K F, Gustafsson L E. Treatment with new organic nitrites in pulmonary hypertension of acute experimental pulmonary embolism. Pharmacol Res Perspect. 2019:e00462.). Investigating this pharmacokinetic principle in several experimental models of acute pulmonary hypertension of various genesis, it has been confirmed that intravenous PDNO at low dosages was an efficient pulmonary vasodilator without provoking systemic hypotension (Nilsson K F, Gustafsson L E. Treatment with new organic nitrites in pulmonary hypertension of acute experimental pulmonary embolism. Pharmacol Res Perspect. 2019:e00462; Nilsson K F, Gozdzik W, Frostell C, Zielinski S, Zielinska M, Ratajczak K, et al. Organic mononitrites of 1,2-propanediol act as an effective NO-releasing vasodilator in pulmonary hypertension and exhibit no cross-tolerance with nitroglycerin in anesthetized pigs. Drug Des Devel Ther. 2018; 12:685-94; Nilsson K F, Gozdzik W, Zielinski S, Ratajczak K, Goranson S P, Rodziewicz S, et al. Pulmonary Vasodilation by Intravenous Infusion of Organic Mononitrites Of 1,2-Propanediol in Acute Pulmonary Hypertension Induced by Aortic Cross Clamping and Reperfusion: A Comparison With Nitroglycerin in Anesthetized Pigs. Shock. 2020; 54(1):119-27, and Stene Hurtsen A, Zorikhin Nilsson I, Dogan E M, Nilsson K F. A Comparative Study of Inhaled Nitric Oxide and an Intravenously Administered Nitric Oxide Donor in Acute Pulmonary Hypertension. Drug Des Devel Ther. 2020; 14:635-45).

Previous animal experimental work has, unexpectedly, indicated that intravenous infusion of PDNO in a low dose (targeted for pulmonary vasodilation) had renal protective and inflammatory dampening effects in a multiorgan failure model (aortic cross clamping and reperfusion) (Nilsson K F, Gozdzik W, Zielinski S, Ratajczak K, Goranson S P, Rodziewicz S, et al. Pulmonary Vasodilation by Intravenous Infusion of Organic Mononitrites Of 1,2-Propanediol in Acute Pulmonary Hypertension Induced by Aortic Cross Clamping and Reperfusion: A Comparison With Nitroglycerin in Anesthetized Pigs. Shock. 2020; 54(1):119-27.). It has also been shown that high dose of intravenous PDNO (targeted for systemic effects) ameliorated kidney function in an ovine model of renal ischemia-reperfusion (Nilsson K F, Sandin J, Gustafsson L E, Frithiof R. The novel nitric oxide donor PDNO attenuates ovine ischemia-reperfusion induced renal failure. Intensive Care Med Exp. 2017; 5(1):29.). In these studies local blood flow was not monitored (only the general cardiac output was monitored) and thus the knowledge about doses required to affect blood flow, especially blood flow in specific organs such as the brain (or even specific sides of the brain), extremities (e.g. one of the legs), kidneys (or a specific kidney) or part of the intestine has not been studied. Nor has the relationship between such changes in flow in relation to effects on the systemic blood pressure and/or cardiac output been measured. The reason for such studies not being performed is they are not a natural continuation of the developmental program for exploring the role of PDNO in the lung blood circulation and the general systemic circulation. The findings regarding these presented herein are astonishing since there was not even any motivation provided by the general development program to investigate this route and with an entirely different focus the previous studies could not be extrapolated to the inventions described herein.

Clinically available NO donor drugs are, therefore, normally administrated intravenously resulting in systemic effects including systemic hypotension, which is the most important dose-limiting side effect. Low systemic blood pressure can be detrimental, especially in critically ill patients, and may counteract the wanted increase in blood flow by severely lowering perfusion pressure (Gresele P, Momi S, Guglielmini G. Nitric oxide-enhancing or -releasing agents as antithrombotic drugs. Biochem Pharmacol. 2019; 166:300-12.).

It is today acknowledged that NO from organic nitrites can be released both non-enzymatically, by acid-catalysed hydrolysis, and enzymatically but the importance of each route for the liberation of NO from organic nitrites, including PDNO, is not established. Since the lung is commonly accepted of being a metabolically active organ one could anticipate that the degradation of PDNO in the lung is considerably more potent than in other organs.

In view of the above, there is a need for improved treatments with NO donors, particularly for conditions where NO has a beneficial effect, that overcomes one or more of the disadvantages described above. Such need is present particularly in organs and parts of the body other than the lungs.

### DESCRIPTION OF THE INVENTION

The present inventors were surprised by the potency and effects of PDNO degradation discovered when intraarterial infusions were tested. That is the inventors were surprised by the fast onset of release of nitric oxide that induces the beneficial effects without having the PDNO passing the pulmonary circulation first. What is even more surprising was that the dose in which PDNO could be given and the magnitude of the response in the vessels of the brain, limbs, kidneys and intestine was unexpected when compared to the dosing possible when administered intravenously in that local treat whilst avoiding, or reducing, systemic effects. The present inventors were also surprised that a lower dose was required by intraarterial administration to achieve an effect versus intravenous administration. The present inventors were further surprised that at higher doses administered intraarterially that did affect the systemic blood pressure these had a pronounced effect on blood flow over the specific target organs, where they had expected the local effect to lessen due to the decrease in driving pressure.

The invention also includes the surprising finding that PDNO in experiments performed could elicit antithrombotic effects. To be able to achieve this NO has to be delivered in a sufficient amount to enter into the thrombocytes. Given that the platelets are surrounded by red blood cells filled with haemoglobin, normally acting as a sink for NO which in blood has been shown to have a biological half-life in the milli second range, it exceptional that PDNO in spite of this seem to affect the platelet function even in considerable low doses.

Whether this means that in from PDNO is released within the platelets or if this is accomplished by another unknown mechanism is unclear. However, the combined targeted local vasodilatory and antithrombotic effects of PDNO is surprising and is of potential great value in treatments of several forms of tissue ischemia.

### Intra-Arterial Administration

The present inventors have unexpectedly found that administering certain mono- and/or bis-nitrosylated propanediols, including compositions and formulations thereof, to a patient in need thereof via intra-arterial infusion at certain doses is able to achieve local, site-specific, treatment of conditions, particularly those conditions where NO is expected to have a beneficial effect, whilst avoiding unwanted systemic side effects.

As shown in the examples, when such compounds are administered intravenously systemic effects are seen at doses much lower than what can be administered intra-arterially before any systemic effect is seen.

Therefore, in a first aspect of the invention there is provided a compound of formula (I):

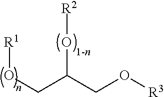

wherein R¹, R² and R³ each independently represent H or —NO,
wherein n is or 1;
wherein when n is 0, R¹ is H; and
wherein when n is 1, R² is H,
provided that at least one of R¹, R² and R³ represents —NO,
for use in the treatment of a condition, wherein the compound of formula (I) is administered to a patient in need thereof via intra-arterial infusion at a dose of from about 0.01 to 3000 nmol kg⁻¹ min⁻¹, which method is referred to herein as “the first method of the invention”.

According to the first method of the inventions there is further provided:
A method of treating a condition by administering a compound of formula (I) to a patient in need thereof via intra-arterial infusion at a dose of from about 0.01 to 3000 nmol kg⁻¹ min⁻¹; and
the use of a compound according to formula (I) for the manufacture of a medicament for the treatment of a condition where the compound is administered to a patient in need thereof via intra-arterial infusion at a dose of from about 0.01 to 3000 nmol kg⁻¹ min⁻¹.

Unless otherwise indicated, all technical and scientific terms used herein have the same meaning as commonly understood by one of ordinary skill in the art to which this invention pertains.

All embodiments of the invention and particular features mentioned herein may be taken in isolation or in combination with any other embodiments and/or particular features mentioned herein (hence describing more particular embodiments and particular features as disclosed herein) without departing from the disclosure of the invention.

As used herein, the term “comprises” will take its usual meaning in the art, namely indicating that the component includes but is not limited to the relevant features (i.e. including, among other things). As such, the term “comprises” will include references to the component consisting essentially of the relevant substance(s).

As used herein, unless otherwise specified the terms “consists essentially of” and “consisting essentially of” will refer to the relevant component being formed of at least 80% (e.g. at least 85%, at least 90%, or at least 95%, such as at least 99%) of the specified substance(s), according to the relevant measure (e.g. by weight thereof). The terms “consists essentially of” and “consisting essentially of” may be replaced with “consists of” and “consisting of”, respectively.

For the avoidance of doubt, the term “comprises” will also include references to the component “consisting essentially of” (and in particular “consisting of”) the relevant substance(s).

The skilled person will understand that references to the treatment of a particular condition (or, similarly, to treating that condition) take their normal meanings in the field of medicine. In particular, the terms may refer to achieving a reduction in the severity of one or more clinical symptoms and/or signs associated with the condition. For example, in the case of pulmonary embolism, the term may refer to achieving reduction in the severity of chest pain, shortness of breath and/or pulmonary hypertension via vasodilation. Furthermore, in the case of pulmonary embolism, the term may also refer to achieving pulmonary vasodilation or a decrease in pulmonary vascular resistance and right ventricular strain.

As used herein, references to patients will refer to a living subject being treated, including mammalian (e.g. human) patients. In particular, the term patient may refer to a human subject. The term patient may also refer to animals (e.g. mammals), such as household pets (e.g. cats and, in particular, dogs), livestock and horses.

The method according to the invention comprises administering to a patient in need thereof an effective amount of a compound according to formula (I). As used herein, the term “effective amount” will refer to an amount of a compound that confers a therapeutic effect on the treated patient. The effect may be objective (i.e. measurable by some test or marker) or subjective (i.e. the subject gives an indication of and/or feels an effect).

The compound of formula (I) may be administered at a dose of from about 0.01 to about 300 nmol kg⁻¹ min⁻¹, for example from about 1 to about 300 nmol kg⁻¹ min⁻¹, such as from about 1 to about 100 nmol kg⁻¹ min⁻¹, or from about 10 to about 3000 nmol kg⁻¹ min⁻¹, such as 10 to about 300 nmol kg⁻¹ min⁻¹.

Preferably, the compound of formula (I) is administered in a dose of from about 1 to about 30 nmol kg⁻¹ min⁻¹. For example, the compound of formula (I) may be administered in a dose of from about 1 to less than about 30 nmol kg⁻¹ min⁻¹, such as about 1 to about 25, 26, 27, 28 or 29 nmol kg⁻¹ min⁻¹, for example from about 1 to about 20 nmol kg⁻¹ min⁻¹, about 1 to about 15, 14, 13, 12 or 11 nmol kg⁻¹ min⁻¹, such as from about 1 to about 10 nmol kg⁻¹ min⁻¹.

The compound of formula (I) may be administered continuously, such as continuous infusion, and this may occur for a time period of up to 14 days, such as 7 days.

The compound of formula (I) may alternatively be administered in a single dose, or in repeated single shots, such as in a bolus infusion.

The compound of formula (I) may be administered to any part of the arterial vasculature that is accessible to the medical professional to deliver the compound of formula (I) by infusion. The infusion may be achieved by a surgical technique, such as through use of a catheter apparatus.

The intra-arterial administration may be with the aim to deliver the compound according to formula (I) to the lower limb and this may be achieved by intra-arterial administration to the femoral artery.

The intra-arterial administration may also, or alternatively, be with the aim to deliver the compound according to formula (I) to the brain and this may be achieved by intra-arterial administration to the carotid artery.

The intra-arterial administration may further, or alternatively, be with the aim to deliver the compound according to formula (I) to the intestine and this may be achieved by intra-arterial administration to the superior mesenteric artery.

The intra-arterial administration may further, or alternatively, be with the aim to deliver the compound according to formula (I) to the kidney, the adrenal gland and/or the ureter and this may be achieved by intra-arterial administration to the renal artery.

The compound of formula (I) may, therefore, be administered via intra-arterial infusion to the femoral artery, the carotid artery, the superior mesenteric artery, the splenic artery, the hepatic artery and/or the renal artery.

When the compound of formula (I) is administered via intra-arterial infusion to the femoral artery and/or the carotid artery, it may be administered at a dose of from about 1 to about 300 nmol kg⁻¹ min⁻¹, such as 1 to about 60 nmol kg⁻¹ min⁻¹, for example 1 to about 50 nmol kg⁻¹ min⁻¹, such as 1 to about 30 nmol kg⁻¹ min⁻¹.

When the compound of formula (I) is administered via intra-arterial infusion to the superior mesenteric artery, it may be administered at a dose of from about 3 to about 3000 nmol kg⁻¹ min⁻¹, such as from about 30 to about 1000 nmol kg⁻¹ min⁻¹, for example from about 100 to about 1000 nmol kg⁻¹ min⁻¹.

When the compound of formula (I) is administered via intra-arterial infusion to the renal artery, it may be administered at a dose of from about 0.01 to about 300 nmol kg⁻¹ min⁻¹, for example from about 1 to about 30 nmol kg⁻¹ min⁻¹.

When administering to a specific artery, this is intended to also include administering to an arterial branch that leads from that artery. For example, when administering to the femoral artery, the administration could be to the superficial epigastric artery, superficial circumflex artery, the external pudendal artery, the deep femoral (or profunda femoris) artery or the superficial femoral artery, which all are connected to the femoral artery.

When administering to the carotid artery, this may be via cerebral arteries, i.e. circulus willisi.

The condition to be treated according to the first method of the invention may be one wherein NO has a beneficial effect. That is to say, the compounds according to formula (I) are useful in the treatment of a condition wherein NO, i.e. administration of NO, has a beneficial effect.

As used herein, the term “beneficial effect” means that the use/administration of the compounds/compositions of the invention leads to an identifiable treatment, and/or improvement, of the condition in the patient being treated. The beneficial effect may be temporary or permanent and may be measured or determined by a medical practitioner or by the patient themselves.

A particular result of the present invention is that the beneficial effect may be experienced locally, e.g. just in one organ of the patient, but not have a clinically significant systemic effect. The beneficial effect may be objective (i.e. measurable by some test or marker) or subjective (i.e. the subject gives an indication of and/or feels an effect).

The condition to be treated may be selected from the group consisting of:
acute pulmonary vasoconstriction of different genesis; pulmonary hypertension of different genesis, including primary hypertension and secondary hypertension; preclampsia; eclampsia; conditions of different genesis in need of vasodilation; erectile dysfunction; systemic hypertension of different genesis; regional vasoconstriction of different genesis; local vasoconstriction of different genesis; acute heart failure (with or without preserved ejection fraction (HFpEF)); coronary heart disease; myocardial infarction; ischemic heart disease; angina pectoris; instable angina; cardiac arrhythmia; acute pulmonary hypertension in cardiac surgery patients; acidosis; inflammation of the airways; cystic fibrosis; COPD; immotile cilia syndrome; inflammation of the lung; pulmonary fibrosis; acute lung injury (ALI); adult respiratory distress syndrome; acute pulmonary oedema; acute mountain sickness; asthma; bronchitis; hypoxia of different genesis; ischemic diseases of different genesis; stroke; cerebral vasoconstriction; inflammation of the gastrointestinal tract; gastrointestinal dysfunction; gastrointestinal complication; IBD; Crohn's disease; ulcerous colitis; liver disease; pancreas disease; inflammation of the bladder of the urethral tract; inflammation of the urinary bladder and ureters of the urethral tract; inflammation of the skin; diabetic ulcers; diabetic neuropathy; psoriasis; inflammation of different genesis; wound healing; organ protection in ischemia-reperfusion conditions; organ transplantation; tissue transplantation; cell transplantation; acute kidney disease; uterus relaxation; cervix relaxation; thromboembolic diseases, including diseases that are complicated by thromboembolism, such as various blood diseases; arterial occlusion, such as arterial embolism; and conditions where smooth muscle relaxation is needed.

In particular, the condition to be treated may be pulmonary hypertension of different genesis, including primary hypertension and secondary hypertension.

Particular conditions that may be include those selected from the group consisting of ischemic diseases of different genesis; thromboembolic diseases including diseases that are complicated by thromboembolism, such as various blood diseases; arterial thrombosis; peripheral ischemia (limb ischemia); thromboembolic stroke; pulmonary embolism; acute mesenteric ischemia (mesenteric arterial occlusion); acute renal artery occlusion; arterial stenosis; arterial occlusion; infarction of the spleen; infarction of the liver, infarction of the lung; Kawasaki disease; and arterial embolism.

With regard to the treatment of ischemic diseases, it is to be understood that this includes the treatment/prophylaxis of conditions arising from arterial occlusions (partial or full), which result in restricted flow to a specific organ and ischemia or threatening ischemia.

The treatment may be manifest by a decline in mean systemic arterial pressure (MAP) of no greater than about 10% relative to the MAP baseline in the subject prior to treatment.

For example, the treatment may be manifest by a decline in MAP of no greater than about 9%, 8%, 7%, or 6% relative to the MAP baseline in the subject prior to treatment. In particular, the treatment may be manifest by a decline in MAP of no greater than about 5% relative to the MAP baseline in the subject prior to treatment.

The treatment may be manifest by a decline in end-tidal NO (ETNO) of no greater than about 10% relative to the ETNO baseline in the subject prior to treatment. For example, the treatment may be manifest by a decline in ETNO of no greater than about 9%, 8%, 7%, or 6% relative to the ETNO baseline in the subject prior to treatment. In particular, the treatment may be manifest by a decline in ETNO of no greater than about 5% relative to the ETNO baseline in the subject prior to treatment.

The treatment may be manifest by an increase in methemoglobin fraction in the blood of no greater than about 10% relative to the methemoglobin fraction baseline in the subject prior to treatment. For example, the treatment may be manifest by an increase in methemoglobin fraction in the blood of no greater than about 9%, 8%, 7%, or 6% relative to the methemoglobin fraction baseline in the subject prior to treatment. In particular, the treatment may be manifest by an increase in methemoglobin fraction in the blood of no greater than about 5% relative to the methemoglobin fraction baseline in the subject prior to treatment.

By “relative to the baseline level” we refer to the comparison between the measured MAP level at the beginning of the study (i.e. prior to administration of the compound of formula (I)) to the MAP level following administration. The baseline level is the level immediately prior to the start of the treatment and is used as a comparator for subsequently measured levels (e.g. immediately following a course of treatment, or at a timepoint following conclusion of a course of treatment). Thus, such a comparison is specific for the subject or group of subjects in question and is not an absolute value.

Further particular conditions that may be treated include bacterial, fungal, viral or parasitic infections.

### Treatment of Thromboembolic Disease

The present inventors have further unexpectedly found that administering certain mono- and/or bis-nitrosylated propanediols, including compositions and formulations thereof, to a patient in need thereof is particularly effective in inhibiting platelet aggregation, which is useful in the treatment of thromboembolic disorders.

Therefore, according to a second aspect of the invention there is provided a compound of formula (I):

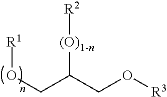

wherein R¹, R² and R³ each independently represent H or —NO,
wherein n is 0 or 1;
wherein when n is 0, R¹ is H; and
wherein when n is 1, R² is H,
provided that at least one of R¹, R² and R³ represents —NO,
for use in the treatment of thromboembolic disease, which is referred to herein as “the second method of the invention”.

According to the second method of the inventions there is further provided:
a method of treating thromboembolic disease by administering a compound of formula (I) to a patient in need thereof; and
the use of a compound according to formula (I) for the manufacture of a medicament for the treatment of thromboembolic disease.

The inhibition of platelet aggregation appears to occur by a surprising method that is different to other treatments and proceeds by inhibition of releasing platelet granula and ATP release.

Thromboembolic diseases are those that are caused when a blood clot that forms in a blood vessel breaks loose, is carried by the bloodstream, and leads to blocking a blood vessel, at least partially.

The thromboembolic disease that is to be treated may be selected from the group consisting of arterial thrombosis; myocardial infarction; cerebral venous thrombosis; portal vein thrombosis; peripheral ischemia (limb ischemia); thromboembolic stroke; pulmonary embolism; acute mesenteric ischemia (mesenteric arterial occlusion); acute renal artery occlusion; arterial stenosis; arterial occlusion; venous thromboembolism, including deep vein thrombosis; and arterial embolism.

Thromboembolic diseases occur both in the venous system and the arterial system and, for the avoidance of doubt, it is envisaged that the use of the compound according to formula (I) in the treatment of such diseases encompasses both venous and arterial treatment. Therefore, the compound according to formula (I) may be administered via intravenous and/or intraarterial infusion.

Furthermore, it is envisaged that the treatment may occur through other routes of administration, such routes include inhalation, nebulisations, intramuscular administration, subcutaneous administration, transdermal administration, intranasal administration, sublingual administration, subconjunctival administration, rectal administration, endotracheal administration, pulmonary administration, gastric administration, ureteral administration, enteral administration, uretheral administration, vesical administration, buccal administration and enteral administration.

The compound according to formula (I) may be administered at a dose of from about 0.01 to 3000 nmol kg⁻¹ min⁻¹, such as from about 0.01 to about 300 nmol kg⁻¹ min⁻¹, for example from about 1 to about 300 nmol kg⁻¹ min⁻¹, such as from about 1 to about 100 nmol kg⁻¹ min⁻¹. Preferably, the compound of formula (I) is administered in a dose of from about 1 to about 30 nmol kg⁻¹ min⁻¹. For example, the compound of formula (I) may be administered in a dose of from about 1 to less than about 30 nmol kg⁻¹ min⁻¹, such as about 1 to about 25, 26, 27, 28 or 29 nmol kg⁻¹ min⁻¹, for example from about 1 to about 20 nmol kg⁻¹ min⁻¹, about 1 to about 15, 14, 13, 12 or 11 nmol kg⁻¹ min⁻¹, such as from about 1 to about 10 nmol kg⁻¹ min⁻¹. Furthermore, the compound of formula (I) may be administered in a dose of from about 1 to about 5 nmol kg⁻¹ min⁻¹.

The compound according to formula (I) may be administered in any way into the vasculature, such as via intra-arterial infusion into any artery of any limb.

The compound according to formula (I) may be administered via intra-arterial infusion, such as to the femoral artery, the carotid arterial, the renal artery or the superior mesenteric artery.

The compound according to formula (I) may be administered via intra-venous infusion, such as to the central venous system, peripheral veins in the arms and legs, femoral veins and scalp veins (particularly scalp veins in neonatals). Intra-venous infusion may also be to deep or superficial veins, such as varicose superficial veins in the leg.

The treatment may be manifest by an increase in methemoglobin fraction in the blood of no greater than about 10% relative to the methemoglobin fraction baseline in the subject prior to treatment. For example, the treatment may be manifest by an increase in methemoglobin fraction in the blood of no greater than about 9%, 8%, 7%, or 6% relative to the methemoglobin fraction baseline in the subject prior to treatment. In particular, the treatment may be manifest by an increase in methemoglobin fraction in the blood of no greater than about 5% relative to the methemoglobin fraction baseline in the subject prior to treatment.

By “relative to the baseline level” we refer to the comparison between the measured MAP level at the beginning of the study (i.e. prior to administration of the compound of formula (I)) to the MAP level following administration. The baseline level is the level immediately prior to the start of the treatment and is used as a comparator for subsequently measured levels (e.g. immediately following a course of treatment, or at a timepoint following conclusion of a course of treatment). Thus, such a comparison is specific for the subject or group of subjects in question and is not an absolute value.

As mentioned above, the inhibition of platelet aggregation appears to occur by a surprising method that is different to other treatments and proceeds by inhibition of releasing platelet granula and ATP release.

Therefore, the second method of the invention also encompasses a method of inhibiting platelet granula release and/or ATP release in a subject, said method comprising administering an effective amount of one or more compounds of Formula (I), or a pharmaceutical formulation thereof, to a subject in need thereof. This aspect of the invention may comprise any of the features outlined above in respect of the treatment of thromboembolic disease.

### Compounds and Compositions

A particular compound for use in both the first and second method of the invention is a compound according to formula (II)

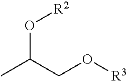

wherein R² and R³ each independently represent H or —NO, provided that at least one of R² and R³ represents —NO.

Two enantiomers of the compound according to formula (II) exist, being the R and S form as depicted below:

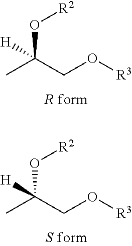

The compounds of formula (I) may contain an asymmetric carbon atom as outlined above and will therefore exhibit optical isomerism.

All stereoisomers and mixtures thereof of the compounds according to formula (I) are included within the scope of both of the invention.

A further particular compound for use in both the first and second method of the invention is a compound according to formula (III):

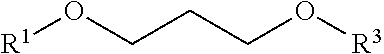

wherein R¹ and R³ each independently represent H or —NO, provided that at least one of R¹ and R³ represents —NO.

A further particular compound for use in both the first and second method of the invention is a compound according to formula (IV):

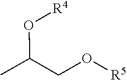

wherein R⁴ and R⁵ each independently represent H or —NO, provided that at least one of R⁴ and R⁵ represents —NO.

The compound for use in both the first and second method of the invention may be present in a composition comprising:
(a) one or more compounds of formula (I) as defined herein; and
(b) one or more corresponding compounds of formula (I) but wherein R¹, R² and R³ represent H (e.g. 1,2-propanediol and/or 1,3-propanediol), which compositions may be referred to hereinafter as “the composition”.

The composition may be substantially non-aqueous. As used herein in relation to both the first and second aspect of the invention, references to “substantially non-aqueous” will refer to the component comprising less than 10%, for example less than 9.9%, 9%, 8%, 7%, 6%, 5%, 4% 3%, 2% or 1% (such as less than 0.5% or less than 0.1%, e.g. less than 0.05%, less than 0.01%) by weight of water.

It is to be understood that the composition may comprise a mixture of compounds falling within formula (I).

Particular compositions that may be mentioned include those wherein the composition comprises from about 0.01% to about 9% (e.g. about 0.01% to about 5%, such as about 3% to about 5%, or about 5% to about 7%) by weight of the one or compound of formula (I).

Particular compositions that may be mentioned include those wherein the composition comprises from about 1 to about 1000 mM (e.g. about 5 to about 750 mM, such as about 5 to about 500 mM, or about 10 to about 203 mM) of the one or more compound of formula (I).

For the avoidance of doubt, the unit mM refers to the concentration of the compound of formula (I) in the composition in 10-3 mol/L and, where the composition comprises a mixture of compounds of formula (I), is based on the average molecular weight of the compounds of formula (I) in the composition.

Particular compositions that may be mentioned include those wherein the composition comprises a compound according to formula (II). Preferably the compound according to formula (II) is the S form.

The S form of the compound according to formula (II) is preferred as this has a higher rate of metabolism, and a different metabolic pathway, than the R form. Furthermore, the S form has a different metabolic degradation route, which results in metabolites which are less toxic than those from the R form.

Particular compositions that may be mentioned include those wherein the composition comprises a compound according to formula (III).

Preferably the compound according to formula (II) is the S form, although it is envisaged that the product is a mixture of both the S and R form of formula (II) with the S form preferably being present in an enantiomeric excess (ee).

In particular embodiments, the compound according to formula (II) may be in an enantiomeric excess of the S form of the compound. That is to say, greater than 50 ee % of the product is in the S form, such as greater than, or equal to, 60 ee %, 70 ee %, 80 ee %, 90 ee %, 95 ee % or 98 ee % of the product is the S form.

In an embodiment where the product is a mono-nitrosylated compound according to formula (II), greater than 50 wt. % of the product is nitrosylated in the 2 position (i.e. R² is —NO), such as between about 55 wt. % and about 80 wt. % is nitrosylated in the 2 position, for example between about 55 wt. % and 75 wt. %.

Particular compositions that may be mentioned include those wherein the composition consists essentially of one or more compounds of formula I and corresponding compounds of formula I but wherein R¹, R² and R³ represent H (i.e. 1,2-propanediol and/or 1,3-propanediol).

Other particular compositions may comprise (or, particularly, consist essentially of or, more particularly, consist of) one or more compounds of formula II and 1,2-propanediol.

Equally, further compositions may comprise (or, particularly, consist essentially of or, more particularly, consist of) one or more compounds of formula III and 1,3-propanediol.

By the term “consist essentially of”, this means that at least 90 wt. % of the defined feature is present, such as at least 95 wt. %, 96 wt. %, 97 wt. %, 98 wt. % or 99 wt. % of the defined feature is present.

Furthermore, particular compositions that may be mentioned include those wherein the composition comprises (or, particularly, consists essentially of or, more particularly, consists of) one or more compounds of formula (II) and (III) along with 1,2-propanediol and 1,3-propanediol.

Particular compositions that may be mentioned include those wherein the composition is substantially free of dissolved nitric oxide.

By the term “substantially free”, this means that the compositions comprise less than 5 wt. %, 4 wt. %, 3 wt. %, 2 wt. % or 1 wt. % of dissolved nitric oxide, such as less than 0.5 wt. % or 0.1 wt./%.

Furthermore, particular compositions may comprise:
(a) one or more compounds of formula IV

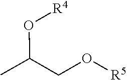

wherein R⁴ and R⁵ each independently represent H or —NO, provided that at least one of R⁴ and R⁵ represents —NO; and
(b) 1,2-propanediol.

The compositions may be administered alone or may be administered by way of known pharmaceutical compositions/formulations.

Accordingly, the composition may be comprised in a pharmaceutical formulation, optionally wherein the pharmaceutical formulation comprises one or more pharmaceutically acceptable excipients.

The skilled person will understand that references herein to pharmaceutical formulations herein refer to the composition in the form of a pharmaceutical formulation and will include references to all embodiments and particular forms thereof.

As used herein, the term pharmaceutically-acceptable excipients includes references to vehicles, adjuvants, carriers, diluents, pH adjusting and buffering agents, tonicity adjusting agents, stabilizers, permeability enhancers, wetting agents and the like. In particular, such excipients may include adjuvants, diluents or carriers.

Particular pharmaceutical formulations that may be mentioned include those wherein the pharmaceutical formulation comprises at least one pharmaceutically acceptable excipient.

Particular pharmaceutical formulations that may be mentioned include those wherein the one or more pharmaceutically acceptable excipients are substantially non-aqueous.

The compound according to Formula (I) may be administered to a patient (i.e. a subject) in conjunction with a suitable aqueous buffer, such as a non-nucleophilic and weakly basic buffer.

More particular embodiments that may be mentioned include those wherein the buffer has a pH of from about 7.1 to about 10 (e.g. about 8 or about 9.2), such as a carbonate (e.g. NaHCO₃) buffer or a phosphate buffer, or a mixture thereof.

In particular, the buffer may be a carbonate buffer with pH 9.2 or a phosphate buffer with pH 8.0 (e.g. a 0.154 molar buffer), or a NaHCO₃ buffer with pH 8.0.

For the avoidance of doubt, references herein to compounds of formula (I) for particular uses may also apply to compositions and pharmaceutical formulations comprising compounds according to Formula (I), as described herein.

Wherever the word ‘about’ is employed herein in the context of amounts, for example absolute amounts, such as weights, volumes, sizes, diameters, etc., or relative amounts (e.g. percentages) of individual constituents in a composition or a component of a composition (including concentrations and ratios), timeframes, and parameters such as temperatures, etc., it will be appreciated that such variables are approximate and as such may vary by ±10%, for example ±5% and preferably ±2% (e.g. ±1%) from the actual numbers specified herein. This is the case even if such numbers are presented as percentages in the first place (for example ‘about 10%’ may mean±10% about the number 10, which is anything between 9% and 11%).

### DESCRIPTION OF THE FIGURES

FIG. 1: Study protocol of dose-response experiments of intravenous and organ-directed intraarterial infusions of 1,2 propanediol organic mononitrites (PDNO) in the carotid and femoral artery (panel A) as well as renal and superior mesenteric artery (panel B).

FIG. 2: Effects of organ-directed intraarterial infusion of 1,2 propanediol organic mononitrites (PDNO) and intravenously administered PDNO on regional blood flow, mean systemic arterial pressure (MAP) and end-tidal nitric oxide concentrations (ETNO) in the left carotid artery (panel A and B) and left common femoral artery (panel C and D). Data are mean with standard error of the mean (SEM). * denotes statistical significance (P<0.05) from baseline (BL) in femoral and carotid intraarterial experiments. #represents statistical significance (P<0.05) from BL during intravenous administration.

FIG. 3: Effects of organ-directed infusion of 1,2 propanediol organic mononitrites (PDNO) and intravenously administered PDNO on regional blood flow, mean systemic arterial pressure (MAP) and end-tidal nitric oxide (ETNO) in the superior mesenteric artery (SMA, panel A and B) and left renal artery (panel C and D). Data are mean with standard error of the mean (SEM). *denotes statistical significance (p<0.05) from baseline (BL) in renal intraarterial experiments and from vasopressin (VP) in SMA experiments. #represents statistical significance (p<0.05) from BL during intravenous administration.

FIG. 4: Dose ratio calculations of end-tidal nitric oxide (ETNO) between intravenous administration of 1,2 propanediol organic mononitrites (PDNO) and intraarterial administration in the left common femoral, left common carotid, left renal and superior mesenteric artery. Dose relations using logarithmized PDNO, ETNO=B₀ e^{B1dose} (panel A) and natural logarithmized ETNO for linearization, In ETNO=B₁ dose+In B₀ (panel B). Estimated coefficients and standard errors (panel C). Significances are shown for comparisons with intravenous (i.v.) ref 1 for carotid intraarterial infusions and femoral intraarterial infusions, and with iv. ref 2 for renal intraarterial infusions and superior mesenteric intraarterial infusions, respectively, unless stated otherwise. ^{a}: Multiple of dose needed to increase log(ETNO) to a similar amount compared to iv. dose. t: intercept for femoral intraarterial infusion compared to carotid intraarterial infusion. #: significant at P<0.001 compared to both renal intraarterial infusions and iv. (ref 2). *: P<0.05, **: P<0.01, ***: P<0.001.

FIG. 5: The arterial fraction of methemoglobin during organ-directed intraarterial infusion of 1,2 propanediol organic mononitrites (PDNO) and intravenously administered PDNO. Data are mean with standard error of the mean (SEM).

FIG. 6: Platelet aggregation in pig whole blood (heparin as anticoagulant) was analysed ex vivo by using Multiplate aggregometer. Coagulated blood clots were administered intravenously to anesthetized pigs (n=5) to pulmonary embolism (PE) and thereafter animals were given intravenous PDNO at 160 nmol/kg/min. Arterial blood was drawn at specific time-points; prior to embolization (baseline), post embolization, during PDNO infusion and after stop of PDNO infusion, and immediately transferred to cuvettes for Mulitplate aggregation analyses. The blood was stimulated with the PAR-1 agonist peptide SFLLRN and platelet aggregation (registered as increases in electrical resistance) was followed over a period of 10 min. Data are individual values and median (the lines). The P-value is from analysis with Dunn's test.

FIG. 7: Anti-aggregatory potency of PDNO. Suspensions of isolated human platelets were activated by the PAR-1 agonist SFLLRN in the absence (open bar) and presence of various concentrations of PDNO (filled bars). Platelet aggregation was assessed as in increases in light transmission in a Chronolog aggregometer. PDNO was introduced to platelets 2 min prior to SFLLRN. The bars represent mean value and standard deviation (n=5).

FIG. 8: Platelet aggregation in whole blood. Suspensions of human blood were stimulated by the PAR-1 agonist SFLLRRN in the absence (open bar) and presence of either PDNO (black bar) or nitroglycerine (grey bar). The NO-donating drugs were introduced to the blood 2 min prior to SFLLRN. Platelet aggregation response was analysed as increases in impedance over a period of 10 min and is expressed as area under the curve. The bars represent mean value and standard deviation (n=4).

FIG. 9: Increases in Ser 239-specific VASP phosphorylation in platelets. Suspensions of isolated human blood platelets were stimulated by either PDNO or nitroglycerine. The platelets were exposed to the NO-donors for 5 min followed by Western immuno blotting for phosphoVASP detection. These experiments were conducted in unstimulated and SFLLRN-stimulated platelets. The PAR-1 agonist was added 2 min after PDNO/nitroglycerine. The bars represent mean value and standard deviation (n=3).

FIG. 10: Study protocol of organ targeted intraarterial infusion of 1,2 propanediol mono-organic nitrites (PDNO, n=12) in the left common femoral artery (CFA), or control state (no infusion, n=12), during six hours post embolization of the CFA.

FIG. 11: Blood flow of the common femoral artery (panel A), femoral vascular resistance (FVR, panel B), femoral venous partial pressure of 02 (FV pO₂, panel C), femoral venous oxygen consumption (FV VpO₂, panel D), femoral venous lactate production (FV Lactate prod, panel E) and delta lactate level between central arterial and femoral venous blood (Delta lactate, panel F) during local arterial infusion of 1,2 propanediol mono-organic nitrites (PDNO, n=12) in the left common femoral artery (CFA), or control state (no infusion, n=12), during six hours post embolization of the CFA. P values indicate the difference between the groups. Data presented as mean±standard error of the mean.

FIG. 12: Mean systemic arterial blood pressure (MAP, panel A) and End-tidal nitric oxide level (ETNO, panel B) during local arterial infusion of 1,2 propanediol mono-organic nitrites (PDNO, n=12) in the left common femoral artery (CFA), or control state (no infusion, n=12), during six hours post embolization of the CFA. Data presented as mean±standard error of the mean.

### EXAMPLES

The invention is illustrated by way of the following examples, which are not intended to be limiting on the general scope of the invention.

### Example 1: Composition

For the studies reported below, a composition comprising a mixture of 1-(nitrosooxy)propan-2-ol and 2-(nitrosooxy)propan-1-ol (which mixture is referred to herein as PDNO) along with 1,2-propane diol (PD; or propylene glycol) was used. The composition contained 6.5 to 7.1 w/w % PDNO, with the balance being propylene glycol.

Processes for preparing the compounds according to formula (I) as defined above, along with compositions comprising these compounds, in particular substantially non-aqueous compositions comprising the compounds and the composition used for the following studies can be found in WO 2020/109420, which is incorporated herein by reference.

For the avoidance of doubt, compounds of formula (I) may also be referred to herein may be referred to by the acronym PDNO, which will indicate that such compounds, including all embodiments and particular features thereof, are used in the methods and uses as described in relation to the present invention. Furthermore, when compositions of PDNO are described that also contain PD, the PD refers to the corresponding propanediol to the compound of formula (I), that is to say the PD is the same compound according to formula (I), but wherein but wherein R¹, R² and R³ represent H.

### Example 2: Intra-Arterial Administration Studies

PDNO is an ultra-fast-releasing with a very short half-life, however, half-life has never been determined. The aim of this study was to compare intraarterial infusion of PDNO with iv infusions regarding local organ blood flow and systemic effects, and to estimate the in vivo half-life of PDNO. A summary of this study is provided below with a detailed methodology following.

Intraarterial infusions of PDNO (0.01-3000 nmol kg⁻¹ min⁻¹) in the left common carotid artery (CCA), left common femoral artery (CFA), left renal artery (RA) and superior mesenteric artery (SMA) were compared to intravenously administered PDNO (3-100 nmol kg⁻¹ min⁻¹) in anesthetized, instrumented pigs (n=14). Intraarterial injections in the left CCA and left CFA and central intravenous injections (PDNO 0.5-4 micromol) were used to estimate in vivo half-life of PDNO in anesthetized, instrumented pigs (n=2). Regional blood flow, mean systemic arterial pressure (MAP), end-tidal nitric oxide (ETNO) and arterial blood gases were measured.

From 1 nmol kg⁻¹ min⁻¹ during the respective intraarterial infusion, the CFA and CCA blood flow was significantly increased from baseline, as was SMA blood flow from 30 nmol kg⁻¹ min⁻¹, whereas RA and iv infusions did not affect blood flow. MAP significantly decreased at 10 nmol kg⁻¹ min⁻¹ during iv infusions, equivalent to 30 and 100 nmol kg⁻¹ min⁻¹ during CCA and CFA infusions, respectively, and to 3000 nmol kg⁻¹ min⁻¹ in the SMA experiments.

The dose equivalents on ETNO between iv administration (reference) and CFA, CCA, RA, and SMA infusion were 2.7, 2.9, 2.6 and 47, respectively. By using the measured circulation times between the intraarterial and intravenous infusion/injection sites, and the dose equivalents from the infusions and injection experiments, the in vivo half-life was estimated to 5.5-6.1 s (infusions) and 3.0-3.9 s (injections).

Organ-directed intraarterial (CCA, CFA and SMA) infusions of PDNO increased organ-specific blood flow dose-dependently, with no or minor systemic effects at effective doses, in contrast to RA and iv infusions. There was a substantial right-shift of the dose-response effects on ETNO concentration when comparing intraarterial and iv infusions probably due to rapid decomposition of PDNO in blood. The in vivo half-life of PDNO was estimated to be in the range of 3.0-6.1 s.

### Study Group

Sixteen domestic 3-month-old pigs (a crossbreed between Swedish country breed, Hampshire and Yorkshire; mean body weight 30 kg [range 27-34 kg], gender ratio 1:1) were included in the study. The experiments were conducted Mar 7-18 and May 9-13, 2022, and Sep. 15, 2023, at the animal laboratory at Örebro University Hospital, Örebro, Sweden. Ethical approval was received from Linköping's regional animal ethics committee (Linköping, Sweden; approval number 00259-2022). The study was conducted in accordance with the Directive 2010/63/EU on the protection of animals used for scientific purposes and followed the ARRIVE guidelines as close as possible.

### Surgical Preparation and Measurements

The animals were anesthetized and ventilated according to a prior report (Stene Hurtsen et. al. A Comparative Study of Inhaled Nitric Oxide and an Intravenously Administered Nitric Oxide Donor in Acute Pulmonary Hypertension, Drug Des Devel Ther. 2020; 14:635-645). Basic instrumentation (venous access for fluids and anesthetics, pulmonary arterial catheter, urinary catheterization) was performed as previously described (Stene Hurtsen et. al. A Comparative Study of Inhaled Nitric Oxide and an Intravenously Administered Nitric Oxide Donor in Acute Pulmonary Hypertension, Drug Des Devel Ther. 2020; 14:635-645). Arterial accesses were gained through ultrasound guided placement of introducers in the brachial arteries bilaterally, 5 Fr (Cordis Corportation, FL, USA) on the right side for access of catheters, and 4 Fr (Cordis Corportation, USA) in the left brachial artery for arterial blood pressure measurement and arterial blood gases.

### Femoral and Carotid Artery Preparation

The femoral arteries and the common carotid arteries were provided with flow probes (3 mm, Transonic System Inc., NY, USA). An angiographic catheter (Soft-Vu Berenstein 5Fr, Queensbury, NY. USA or SOFT-VU Sos Omni® selective 5 Fr, Queensbury NY USA) was positioned in the left carotid or left femoral artery via the 5 Fr introducer (Cordis Corportation, USA) in right brachial artery using a guide wire (Terumo Radiofocus® STIFF type angles 0.035, Terumo corporation TOKYO, Japan). Location was confirmed with angiography. A microcatheter (Terumo Progreat®, 2.7 Fr. Terumo cooperation, Tokyo, Japan) was inserted in the angiographic catheter and placed proximal of the flow probes.

The angiographic catheter was then withdrawn to the brachiocephalic trunk in the carotid experiments and to the external iliac artery in the femoral experiments. In the femoral experiments, a venous peripheral access was gained in a superficial vein on the distal lower limb (BD venflon pro safety, 0.9 mm, Switzerland).

In two animals, PDNO was repeatedly injected via the microcatheter in the left common femoral (1-4 micromol), left carotid artery (0.5-2 micromol) and the CVC in superior caval vein (0.5-4 micromol). In addition, 10 ml of 5% sodium bicarbonate solution was injected at these sites.

### Renal Artery and SMA Preparation

The renal arteries were provided with 3 mm flow probes (Transonic System, USA) through a retroperitoneal incision and the SMA was enclosed with a 6 mm flow probe (Transonic System, USA) through laparotomy. The targeted arteries were provided with a microcatheter (Rebar™-18 Micro Catheter, Microtherapeutics, Inc., CA, USA) in a similar manner as previously described, with the angiographic catheter withdrawn to the aorta in both renal and SMA experiments.

After instrumentation, 5000E heparin (Leo Pharma A/S, Denmark) was given and an intervention free hour followed. The animals were covered with heat blankets aiming for normal body temperature. After the experiments the animals received an overdose of propofol followed by 20 mL Potassium chloride (2 mM, B. Braun Medical Inc. PA, USA) euthanasia was confirmed by absence of systolic blood pressure and ETCO₂.

### Femoral and Carotid Experiments

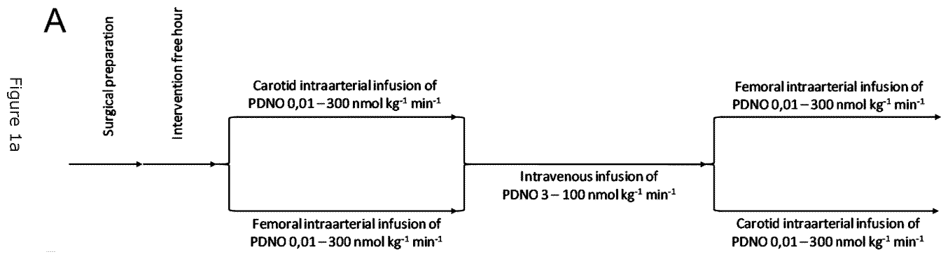

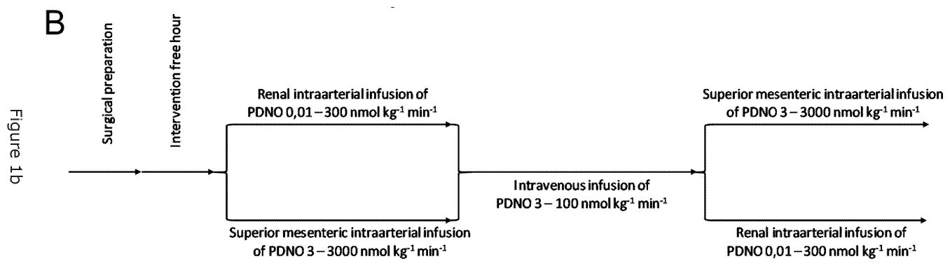

PDNO was infused using syringe pumps with a carrier solution (bicarbonate 1.4%) and administered via the microcatheter at eight doses (0.01, 0.1, 1, 3, 10, 30, 100 and 300 nmol kg⁻¹ min⁻¹, 20 minutes per dose) in the left common femoral and left carotid artery, block randomization was applied (FIG. 1A (carotid and femoral artery) and FIG. 1B (renal and superior mesenteric artery)). Between the local intraarterial infusion routes, intravenous infusions in the central venous catheter at four doses (3, 10, 30 and 100 nmol kg⁻¹ min⁻¹, 10 minutes per dose) were given for reference. There was a washout period of 60 minutes between the PDNO administration.

### Renal Artery and Superior Mesenteric Artery (SMA) Experiments

Eight doses of PDNO (0.01, 0.1, 1, 3, 10, 30, 100 and 300 nmol kg⁻¹ min⁻¹, 20 minutes per dose) were given in the left renal artery through the microcatheter. In the SMA, seven doses (3, 10, 30, 100, 300, 1000 and 3000 nmol kg⁻¹ min⁻¹, 20 minutes per dose) were during concurrent infusion of vasopressin (Empressin®, OrPha Swiss, Switzerland), 0.06 E kg⁻¹ min⁻¹ (40 IU 2 ml⁻¹). Intravenous infusions were given between the local intraarterial infusions with a washout period of 60 minutes in between.

### Data Collection

Hemodynamic and ventilation parameters, including local blood flow in the target organ and fraction of exhaled nitric oxide (FENO; CLD 77, ECO PHYSICS, Switzerland) were continuously recorded (Acqknowledge Software, BIOPAC® Systems Inc, CA, USA). Arterial blood gas analyses were collected at the end of all doses (GEM5000, Werfen, MA, USA).

### Data Analysis and Statistics

Primary outcome was effects on regional blood flow, systemic blood pressure and end-tidal nitric oxide (ETNO). These data are presented as means with standard error of the mean (SEM). Shapiro-Wilk normality test was used to determine normal distribution and linear mixed model was performed to evaluate change from baseline using SPSS (version 27, IBM Corp., Armonk, NY, USA) and graphs were created in GraphPad Prism 9.4.1 (GraphPad Software, Inc., San Diego, CA, USA). Secondary measures were hemodynamic and blood gas measurements. Dose comparison of intraarterial versus intravenous were made. Linear mixed models were fitted where fixed effects were route of administration (categorical), dose (continuous) and the interaction between dose and route, and random effects were subject ID (Ine from n/me v.3.1-16 using R 4.2.2(16). For comparison of doses, estimated coefficients (estimated using the Istrends from emmeans v.1.8.4-1 were compared using the ANOVA function and adjusted using Tukey's HSD test. The circulation time from the intraarterial injection/infusion site (carotid and femoral) to the intravenous injection/infusion sites were calculated by subtracting the time to first exhalation with increase in ETNO and end-tidal CO₂ when PDNO or sodium bicarbonate respectively, were injected in the intraarterial catheter and the central intravenous catheter. In vivo half-life was then estimated by linear regression analysis (In N(t)=k×t+In (N(0)) and T_{1/2}=In 2/k). A P-value<0.05 was considered statistically significant.

### Results on Hemodynamics

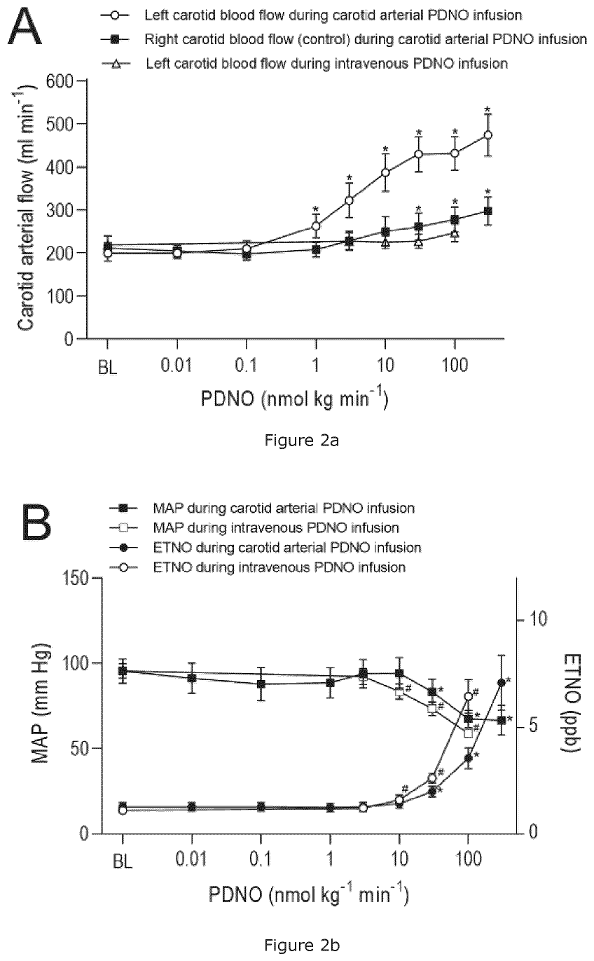

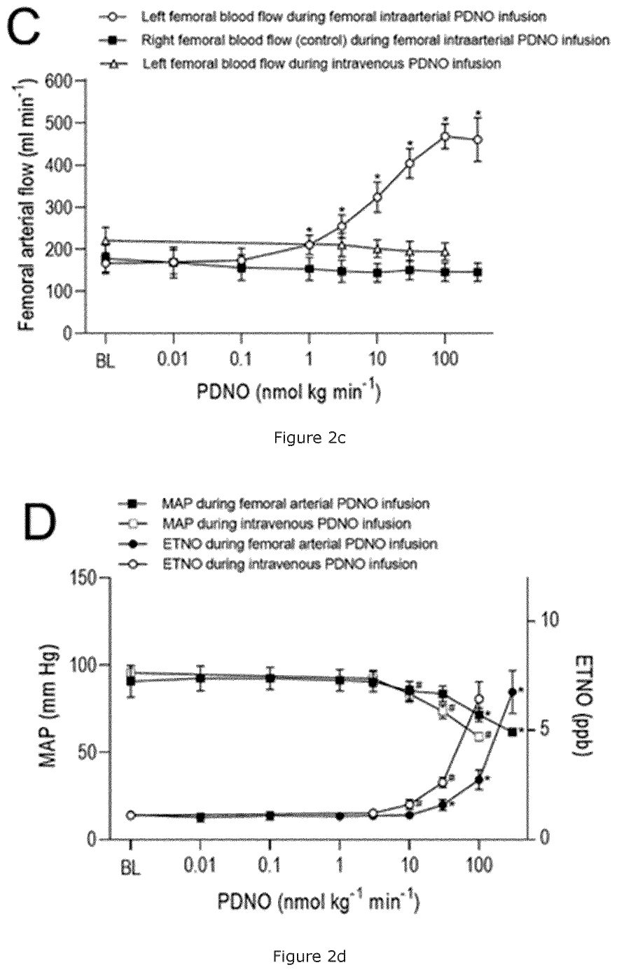

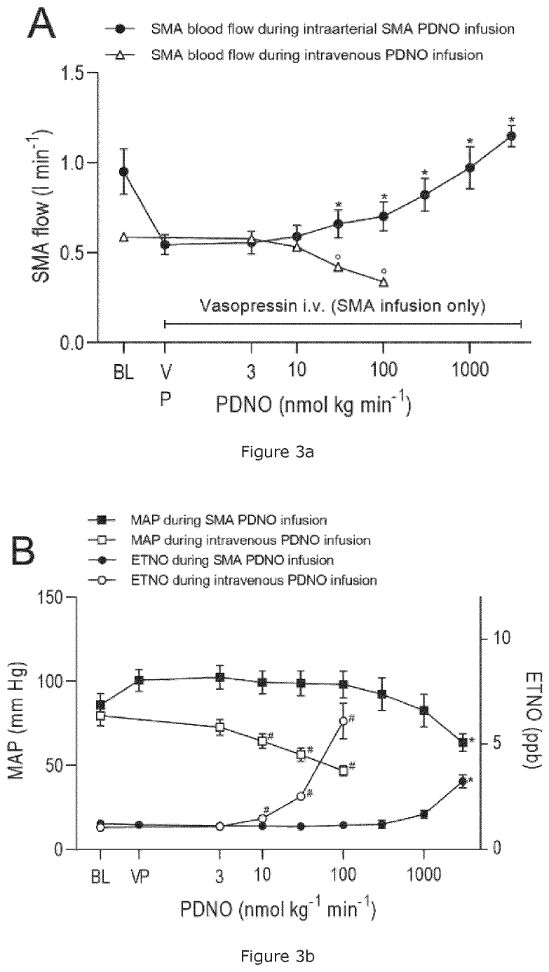

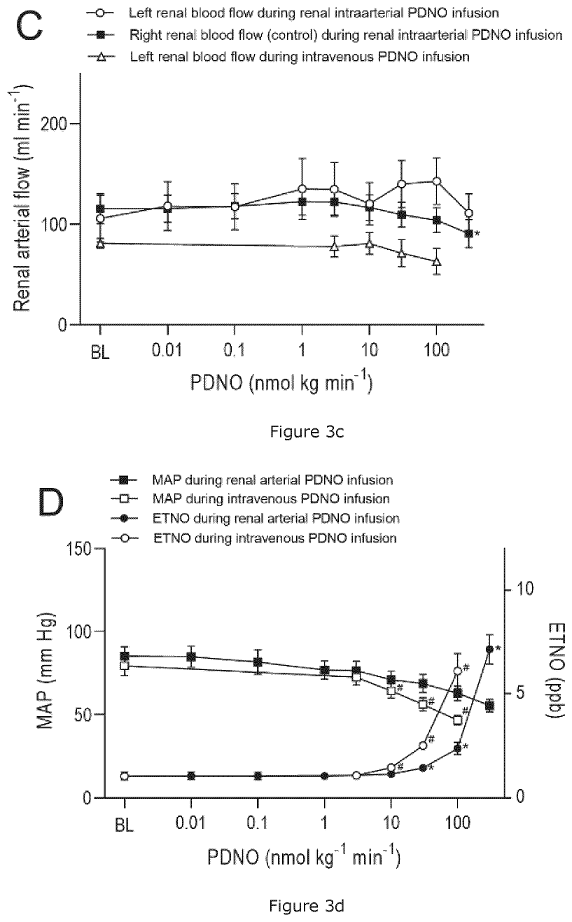

Left femoral and left carotid blood flow started to increase at 1 nmol kg⁻¹ min⁻¹ and the SMA blood flow at 30 nmol kg⁻¹ min⁻¹ during intraarterial PDNO administration at the respective infusion site (P<0.05 compared with baseline, FIGS. 2A and 2B (left carotid), FIGS. 2C and 2D (left femoral), and FIGS. 3A and 3B (SMA)). The contralateral (i.e. right) artery was unaffected in the femoral experiments but significantly increased at above 30 nmol kg⁻¹ min⁻¹ in the carotid experiments (P<0.05, FIG. 2A). The mean systemic arterial pressure (i.e. MAP) started to decrease at 30 and 100 nmol kg⁻¹ min⁻¹ in the carotid and femoral experiments, respectively (P<0.05, FIGS. 2B and 2D respectively), and at 3000 nmol kg⁻¹ min⁻¹ in the SMA experiments (P<0.05, FIG. 3B). Corresponding effect on MAP by intravenous administered PDNO was seen at 10 nmol kg⁻¹ min⁻¹(P>0.05, FIGS. 2B, 2D, 3B and 3D). Cardiac output was unchanged during the experiments except for the last three doses in the carotid experiments (P<0.05). These results surprisingly show that intra-arterial administration of PDNO can be done at higher doses than intra-venous administration to achieve an increased local treatment efficacy without having an overall systemic effect. Furthermore, unexpectedly, the intravenous PDNO infusion did not affect organ blood flow, in contrast to the intra-arterial infusion, indicating the need for the intraarterial infusion route to effectively treat systemic organs.

### ETNO

End-tidal NO (ETNO) started to increase at 30 nmol kg⁻¹ min⁻¹ in the femoral, carotid and renal experiments (P<0.05 compared with baseline) and at 3000 nmol kg⁻¹ min⁻¹ in the SMA experiments (P<0.05 compared with baseline, FIGS. 2B, 2D, 3B and 3D). The corresponding increase in the intravenous experiments was in at 10 nmol kg⁻¹ min⁻¹ (P<0.05 compared with baseline, FIGS. 2 and 3).

The estimated coefficient, and thus the estimated dose-ratio for equivalent effects compared with intravenous infusion, between administration routes using logETNO was 2.7, 2.9, 2.6 and 47 for femoral, carotid, renal and SMA infusion, with intravenous PDNO infusion as a reference (FIG. 4).

These results correspond to those exhibited on the hemodynamics.

### Arterial Blood Cases

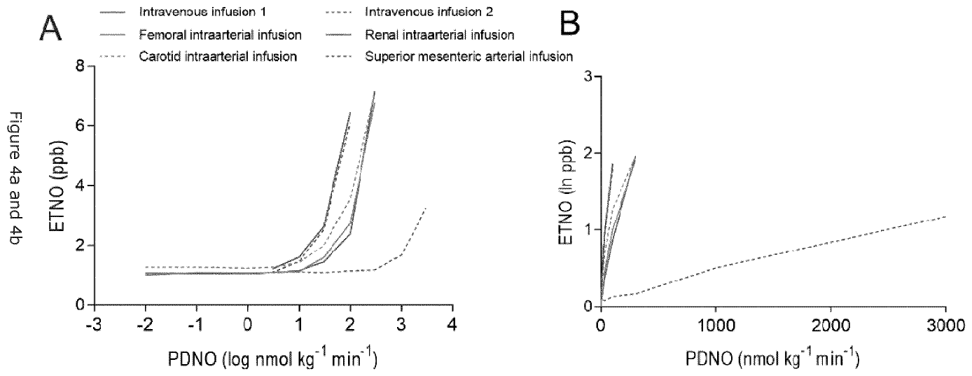

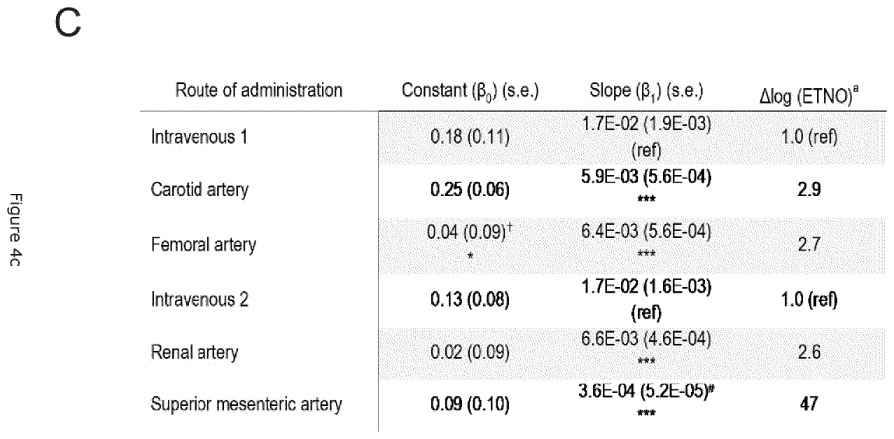

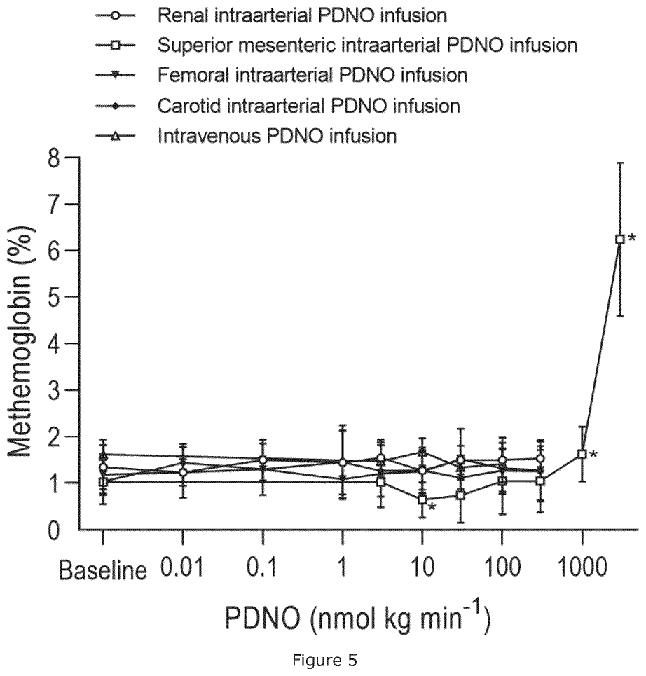

The methemoblobin fraction was significantly higher in the SMA experiments at the two highest doses compared to baseline (P<0.05, FIG. 5). The other administration routes did not change the methemoglobin fraction, which is also particularly surprising and unexpected. The SaO₂ decreased significantly at 30 and 100 nmol kg⁻¹ min⁻¹ in the carotid and femoral experiments, respectively (P<0.05 compared with baseline).

### Intraarterial-to-Intravenous Circulation Times and Half-Life Estimations

The median (25-75 percentiles) circulation times from the carotid and femoral intraarterial injection sites were to first exhalation with increased ETNO 13.5 s (12.3-15.4, n=13) s and 13.7 s (12.9-15.2, n=9), respectively, and from the central intravenous injection site 5.0 s (4.3-5.9, n=29) and 5.3 s (3.3-5.8, n=9). The circulation times between the carotid and femoral intraarterial injection sites to the central intravenous injection site were calculated to 8.5 s and 8.4 s as depicted in Table 1 below. By using the increase in ETNO integral provoked by the intraarterial and central intravenous PDNO injections and the circulation times from each injection site in linear regression analysis, assuming first order kinetics, the summarized half-life of PDNO in vivo was estimated to 3.6 s (3.0-4.4, 95% CI, see Table 2 below for the half-life estimation at each dose). Using the dose-ratio equivalents estimated in the carotid and femoral intraarterial and central intravenous infusion experiments (FIGS. 4A and 4B) in the same equation estimated the half-life of PDNO in vivo to a mean of 5.8 s.

TABLE 1
Half-life estimation derived from the injection and infusion
experiments with PDNO in two anesthetized pigs. Circulation
time between the intraarterial and intravenous injection site
was also estimated with injection of 10 ml 5% sodium bicarbonate
solution. Dose-ratio (DR) was derived from the experiments with
intraarterial and intravenous infusions (FIGS. 1 to 5).

| | | |
|---|---|---|
| | Circulation time | Half-life |
| Injection/Infusion | (median, s) | (s, 95% CI) |

Carotid artery and intravenous

| | | |
|---|---|---|
| Sodium bicarbonate | 7.0 | |
| PDNO | 8.5 | |
| 0.5 micromol | | 3.1 (2.5-4.0) |
| 1 micromol | | 3.4 (2.8-4.5) |
| 2 micromol | | 3.8 (3.4-4.2) |
| All doses included | | 3.7 (2.9-5.3) |
| PDNO DR | | 5.5 |

Femoral artery and intravenous

| | | |
|---|---|---|
| Sodium bicarbonate | 8.9 | |
| PDNO | 8.4 | |
| 1 micromol | | 3.9 (3.4-4.5) |
| 2 micromol | | 3.1 (2.5-4.3) |
| 4 micromol | | 3.0 (2.8-3.1) |
| All doses included | | 3.4 (2.7-4.6) |
| PDNO DR | | 6.1 |

### Conclusions

Organ-directed intraarterial (carotid, femoral and superior mesenteric) infusions of PDNO increased organ-specific blood flow dose-dependently in contrast to renal arterial and intravenous infusions. The effective dose needed for increased carotid, superior mesenteric and femoral blood flow had no or minor effects on systemic blood pressure. There was a substantial right-shift of the dose-response effects on end-tidal NO concentration when comparing intraarterial and intravenous probably due to rapid decomposition of PDNO in blood. Methemoglobin levels were lower than 3% during intraarterial organ directed infusion in doses up to 300 nmol kg⁻¹ min⁻¹. The half-life of PDNO in vivo varied slightly depending on the experimental setup and was estimated to be in the range of 3.0 to 6.1 s.

### Example 3: Anti-Platelet Aggregatory Effects of Intravenous PDNO Administration

### Method

### Study Group

Five domestic 3-month-old pigs (a crossbreed between Swedish country breed, Hampshire and Yorkshire) were used. Free access to food and water was provided until morning of experiments. Ethical approval was received from Linköping's regional animal ethics committee (Linköping, Sweden; approval number 00259-2022). The experiments were conducted at the animal laboratory at Örebro University Hospital in Örebro, Sweden, and followed the Directive 2010/63/EU on the protection of animals used for scientific purposes.

### Animal Preparation and Monitoring

Anesthesia and ventilation were established and maintained according to Stene Hurtsen et al (Stene Hurtsen et. al. A Comparative Study of Inhaled Nitric Oxide and an Intravenously Administered Nitric Oxide Donor in Acute Pulmonary Hypertension, Drug Des Devel Ther. 2020; 14:635-645) except that premedication was changed to 0.05 ml kg⁻¹ of a mix of Zoletil forte (50 mg ml⁻¹ of each) and Medetomidin (1 mg ml⁻¹, Domitor Vet). Basic instrumentation (arterial line, pulmonary arterial catheter, urinary catheterization) was performed as previously described (Stene Hurtsen et. al. A Comparative Study of Inhaled Nitric Oxide and an Intravenously Administered Nitric Oxide Donor in Acute Pulmonary Hypertension, Drug Des Devel Ther. 2020; 14:635-645). Through the arterial line 5 ml kg⁻¹ blood was drawn into 50 ml syringes, coagulation in the syringes was boosted with an intermediate dose of thrombin (0.1 U ml⁻¹) for later pulmonary embolization. A 20 Fr urinary catheter was inserted in the right jugular vein for injection of the coagulated blood. Venous access for fluids and anesthetics was gained through a 7 Fr introducer (Cordis Corportation, USA) in the left external jugular vein. Nitric oxide (NO) in exhaled air was measured through the endotracheal tube (Eco physics, Dürnten, Switzerland). The animals were covered with heat blankets aiming for normal body temperature. After the experiments the animals received an overdose of propofol followed by 20 mL Potassium chloride (B. Braun Medical Inc. PA, USA) euthanasia was confirmed by ECG and ETCO2.

### Experimental Protocol

After the intervention free hour, pulmonary embolism was induced by injection of the coagulated blood in the right external jugular vein to a target mean pulmonary arterial pressure of 45-55 mmHg. In parallel, an intravenous norepinephrine infusion was started and titrated to maintain mean systemic arterial pressure above 60 mmHg. Fraction of inspired oxygen (FiO₂) was increased to maintain normal arterial oxygenation and respiratory frequency was increased to adjust for part of the hypercapnia. After approximately 15 min, an intravenous infusion of PDNO at 160 nmol kg⁻¹ min⁻¹ was started and continued for approximately 15 min and then it was discontinued. After the experiments, the animals were euthanized with a fast intravenous injection of 40 mM potassium chloride after a bolus dose of 200 mg of propofol. Asystole was confirmed with hemodynamic and respiratory measurements.

Hemodynamic and ventilation parameters including end-tidal NO concentration were continuously recorded (Acqknowledge Software, BIOPAC® Systems Inc, CA, USA).

Arterial and mixed venous blood samples were collected at baseline (before pulmonary embolization), after pulmonary embolization, during PDNO 160 nmol kg⁻¹ min⁻¹ and 15 min after discontinuation of PDNO infusion. Arterial and mixed-venous blood samples were analysed in a blood gas machine (GEM5000) and arterial blood samples collected in heparinized syringes were immediately transferred to cuvettes for Multiplate aggregation analyses. The blood was stimulated with the PAR-1 agonist peptide SFLLRN and platelet aggregation (registered as increases in electrical resistance) was followed over a period of 10 min, and calculated as area under the curve (see the methods in example 4 for details).

### Statistical Analyses

The primary outcome was change in PAR-1 agonist peptide SFLLRN induced platelet aggregation between the measurement points pulmonary embolization and intravenous PDNO infusion (by a pairwise comparison [Dunn's test] if Friedman test including all four time points found a P-value<0.05). Secondary outcomes were change in hemodynamic variables, end-tidal NO and blood gas values comparing pulmonary embolization with and without PDNO infusion. No statistical analyses were done on secondary outcomes.

### Results

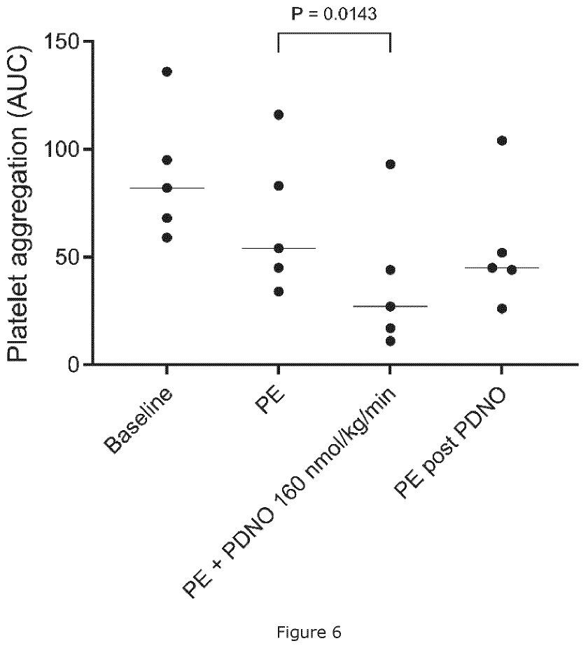

Animals had normal hemodynamic and respiratory variables before pulmonary embolization. Pulmonary embolization induced acute pulmonary hypertension with systemic hypotension (which was reversed with norepinephrine infusion), arterial deoxygenation (which was reversed by increasing FiO₂), hypercapnia (which was partly reversed by increasing respiratory frequency) and increased end-tidal NO. Intravenous PDNO infusion caused decreased pulmonary and systemic vascular resistance as well as increased end-tidal NO; effects that were reversed when stopping the PDNO infusion. Intravenous PDNO infusion during pulmonary embolization statistically significantly decreased PAR-1 agonist peptide SFLLRN induced platelet aggregation compared with before PDNO infusion (P=0.0143, FIG. 6).

### Conclusions

The data shows that intravenous PDNO during pulmonary embolization surprisingly inhibits platelet aggregation (measured ex vivo) and is an effective treatment for thromboembolic diseases. Combined with the local effect achieved through administration of PDNO, such as vasodilation, this invention could further offer an improved treatment for thromboembolic diseases.

### Example 4: Further Anti-Thrombotic Studies

### Platelet Aggregation in Whole Blood

Potential anti-thrombotic effect of PDNO in vitro was evaluated by analysing aggregation responses to the thrombin mimetic hexapeptide SFLLRN. This peptide is an agonist towards the main thrombin receptor designated protease-activated receptor-1 (PAR-1). Inhibitory effects of PDNO were elucidated in isolated suspensions of human platelets and in whole blood.

Heparinized human or pig blood (0.5 ml) was diluted 1:1 with Krebs-Ringer glucose (KRG) buffer (isotone saline, pH 7.4). The diluted blood was transferred into analyse cuvettes and placed in a Multiplate aggregometer. Platelet aggregation in vitro (human blood) and ex vivo (pig blood) was measured as increase in electrical impedance between two platinum electrodes. Measurements were conducted at 37 °C under stirring conditions.

In the in vitro experiments, PDNO or nitroglycerine (used as control drug) was introduced 2 min prior to the hexapeptide SFLLRN. This hexapeptide acts as agonist to the main thrombin receptor designated protease-activated receptor-1 (PAR-1). In the ex vivo experiments, PDNO was given intravenously to anaesthetized pigs. After blood drawing, diluted blood was transferred to the Multiplate instrument and stimulated by SFFLRN. Aggregation responses were registered during 10 min and expressed as area under curve.

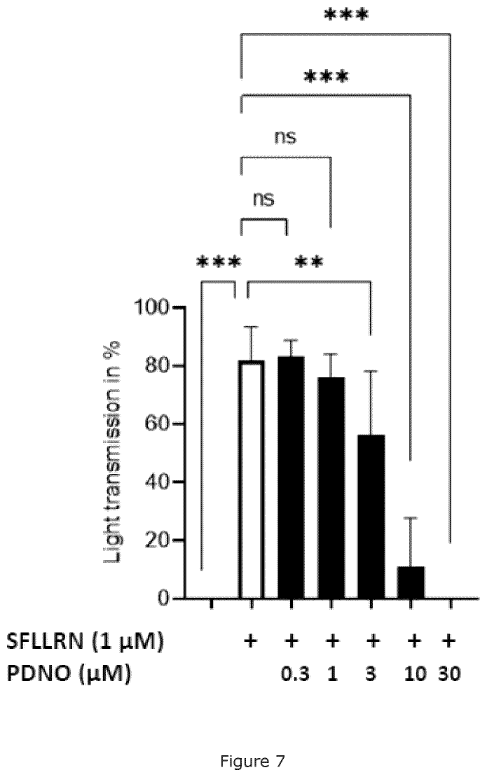

By measuring increases in light transmission (Born aggregometry) in aliquots of isolated platelets following addition of SFLLRN, potential anti-aggregatory activity of PDNO was investigated. As shown in FIG. 7, PDNO concentration-dependently inhibited SFLLRN-induced platelet aggregation indicative for an antithrombotic activity.

### Aggregation of Isolated Platelets

Platelet aggregation in whole blood (human blood with heparin as anticoagulant) was analysed as increases in electrical resistance (impedance changes between two platinum electrodes) using a Multiplate aggregometer.

Heparinized blood was obtained from healthy volunteers and immediately mixed with an acid citrate dextrose (ACD) solution at a volumetric proposition of 5 parts blood and 1 part of ACD. The platelets were centrifuged for 20 min at 220×g to obtain platelet-rich plasma.

Thereafter, platelets were pelleted by a second centrifugation (20 min at 480×g) and gently resuspended in KRG buffer. 0.3 ml of isolated platelet suspension (2.5×108 platelets/ml) was placed in a Chrono-log aggregometer and analyses were conducted at 37 degrees under stirring conditions. Platelets were stimulated with PDNO or nitroglycerine for 2 min and thereafter activated by SFLLRN. Platelet aggregation was assessed as % increase in light transmission through the cuvettes (light transmission in platelet-free KRG represent 100%).

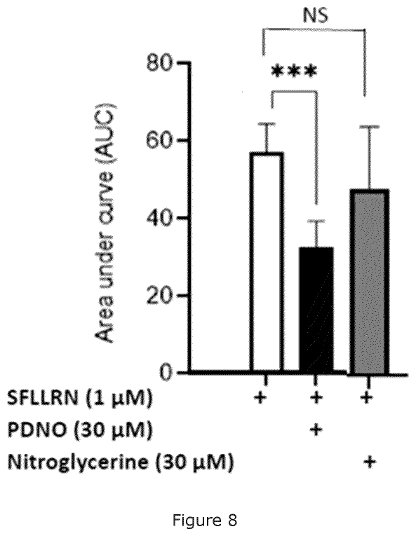

FIG. 8 shows that PDNO, but not nitroglycerine significantly inhibited SFLLRN-induced platelet aggregation under these experimental conditions. This finding indicates that PDNO produces antithrombotic actions even in whole blood and in the presence of haemoglobin.

### Detection of Protein Phosphorylation by Western Blotting

In platelets and vascular smooth muscle cells, the cytoskeleton-binding protein vasodilator-stimulated phosphoprotein (VASP) is a main molecular target for NO/cyclic GMP signalling pathway. Ser239 specific phosphorylation of VASP following NO exposure of platelets is believed to be a pivotal molecular mechanism underlying anti-aggregatory/antithrombotic effects of NO. The effect of PDNO on Ser239-specific VASP phosphorylation was analysed by Western (immuno) blotting.

Aliquots of isolated platelet suspensions were exposed to either PDNO or nitroglyceine for 5 min. This series of experiments were conduced both on resting and SFLLRN-stimulated platelets.

Specifically, NO/cyclic GMP-induced phosphorylation of Ser239 on vasodilator-stimulated phosphoproteins (VASP) was detected by Western blotting. Isolated suspensions (0.2 ml) of platelets were stimulated by PDNO or nitroglycerine for 5 min in the absence or presence SFLLRN. The hexapeptide was added 2 min after the NO-donors. Platelets were lysed by adding 50 μl of sodium-dodecyl sample buffer and proteins were further denatured at 95 °C for 5 min. Platelet proteins were separated by electrophoresis and thereafter blotted onto polyvinylidene fluoride membranes. For determining phosphorylation of VASP, membranes were incubated by Ser239-specific VASP antibodies followed by horseradish peroxidase antibodies and visualized in a Fuji LAS Chemiluminometer.

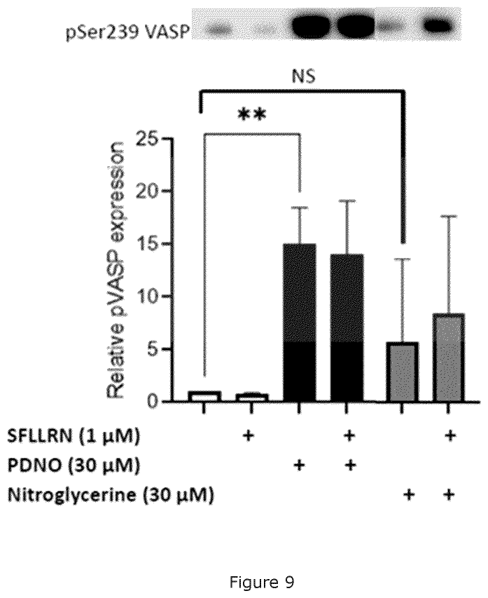

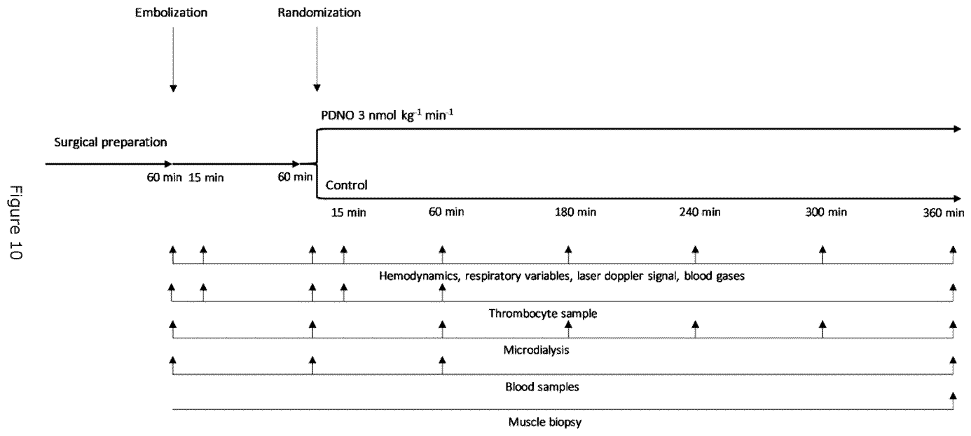

As shown in FIG. 9, PDNO but not nitroglycerine provoked a significant increase in Ser239-specific VASP phosphorylation. These data show that PDNO induces prominent NO/cyclic GMP signalling in human platelets and that this is associated with significant anti-aggregatory effects (FIGS. 7 and 8).

### Example 5: Intraarterial Infusion of PDNO to Treat Acute Lower Limb Embolisation

### Study Group

Twenty-four domestic 3-month-old pigs (a crossbreed between Swedish country breed, Hampshire and Yorkshire (gender ratio 1:1, weight 32±4 kg)) were used. Free access to food and water was provided until morning of experiments. Ethical approval was received from Linköping's regional animal ethics committee (Linköping, Sweden; approval number 00259-2022). The experiments were conducted Sep. 12-30, 2022 and Jan. 24-31, 2023, at the animal laboratory at University hospital in Örebro, and followed the Directive 2010/63/EU on the protection of animals used for scientific purposes.

### Animal Preparation and Monitoring

Anesthesia and ventilation were established and maintained according to Stene Hurtsen et al (Stene Hurtsen et. al. A Comparative Study of Inhaled Nitric Oxide and an Intravenously Administered Nitric Oxide Donor in Acute Pulmonary Hypertension, Drug Des Devel Ther. 2020; 14:635-645) for the first 14 animals, for the last 10 animals premedication was changed to 0.05 ml kg⁻¹ of a Zoletil forte (250 mg+250 mg) and Medetomidin (1 mg ml⁻¹, Domitor Vet) mix. Basic instrumentation (arterial line, pulmonary arterial catheter, urinary catheterization) was performed as previously described (Stene Hurtsen et. al. A Comparative Study of Inhaled Nitric Oxide and an Intravenously Administered Nitric Oxide Donor in Acute Pulmonary Hypertension, Drug Des Devel Ther. 2020; 14:635-645). Through the arterial line 1 ml kg⁻¹ blood was withdrawn through 5 ml syringes (Cordis Medical ApS, Denmark) for later embolization. Venous access for fluids and anesthetics was gained through a 7 Fr introducer (Cordis Corportation, USA) in the left external jugular vein. A 5 Fr catheter (Soft-Vu Berenstein 5Fr, Queensbury, NY, USA) was placed through the left venous access and advanced to the common femoral vein at the bifurcation to the profunda vein. Likewise, a 5 Fr catheter (Soft-Vu Berenstein, USA) was inserted through the brachial artery via a 5 Fr (Cordis Corportation, USA) introducer and advanced to the common femoral artery (CFA). Positions confirmed by fluoroscopy. A flow probe (Transonic Systems Inc. NY, USA) was placed on the CFA bilaterally, 3 mm on the left side, 6 mm on the right side. A laser doppler probe (Perimed, Sweden) was placed in the peroneus muscles of the hind limb and a microdialysis catheter (M Dialysis AB, Sweden) was placed in the anterior tibial muscle. Nitric oxide in exhaled air was measured through the endotracheal tube (Eco physics, Dürnten, Switzerland). The animals were covered with heat blankets aiming for normal body temperature. After the experiments the animals received an overdose of propofol followed by 20 mL Potassium chloride (B. Braun Medical Inc. PA, USA) euthanasia was confirmed by ECG and ETCO₂.

### Experimental Protocol

After the intervention free hour the left common femoral artery was selectively embolized through the 5 Fr catheter (Berenstein, USA) with autologous blood clots 1 ml kg⁻¹ (clotting time at a minimum of 2 hours) 5 ml min⁻¹. One hour after embolization block randomization to either treatment with PDNO 3 nmol kg⁻¹ min⁻¹ or control group (no infusion) was performed (FIG. 10). The NO donor was administered through syringe pumps with a carrier solution (bicarbonate 1.4%) through a microcatheter (Terumo Progreat®, 2.7 Fr. Terumo cooperation, Tokyo, Japan) inserted through the 5 Fr catheter (Berenstein, USA) in the right brachial artery and directed to the common femoral artery. The 5 Fr catheter (Berenstein, USA) was pulled out to the aortic bifurcation in both groups. Reperfusion time followed for six hours. At the end of the protocol a muscle biopsy of the anterior tibial muscle was taken bilaterally and put in formalin (Solveco AB, Rosersberg, Sweden) for blinded histopathological evaluation.

Hemodynamic and ventilation parameters, including local blood flow in the CFA was continuously recorded (Acqknowledge Software, BIOPAC® Systems Inc, CA, USA). Laser doppler signal, fraction of exhaled nitric oxide (FENO, apparaten), arterial and local venous blood samples and blood gases and were collected according to protocol (FIG. 10).

Primary outcome was blood flow of the left femoral artery. Secondary outcomes were ischemia index score after six hours (concentration of lactate, potassium, aspartate aminotransferase, lactate dehydrogenase and creatine kinase (Ref 19667877)), lower limb microcirculation (via laser doppler), histopathology of a lower limb muscle biopsy. Side effects were measured (methemoglobinemia, systolic blood pressure). Measurements were collected according to FIG. 10.

### Calculations

Femoral venous resistance (FVR): Femoral arterial blood flow/MAP.

Delta lactate (Δ Lactate): Central arterial lactate level−femoral venous lactate level.

Lactate production: Femoral arterial blood flow×Δ Lactate×−1.

Central arterial and femoral venous saturation levels: (partial pressure of oxygen[pO₂]2.94)/(pO22.94+(4.76×(10(−0.441×(pH−7.4))))\hat{ }2.94).

Central arterial and femoral venous content of O₂: ((saturation level×Hb)/10×1.31+0.025×pO₂)×10.

Femoral venous O₂ consumption: (arterial content of O₂−femoral venous content of O₂)×(arterial femoral blood flow/1000).

### Statistical Analysis

Normal distribution was analyzed with Shapiro-Wilk test. Normally distributed data were analyzed using linear mixed model, with group and time as main factors and their interaction followed by multiple interactions. Normal distributed data are presented with mean±standard error of the mean. Data deviating from normal distribution were analyzed using Mann Whitney U test and presented as median (IQR 25th-75% percentile). All statistics were performed in SPSS (version 27, IBM Corp., Armonk, NY, USA) and graphs were generated in GraphPad Prism 9.4.1 (GraphPad Software, Inc., San Diego, CA, USA).

### Results

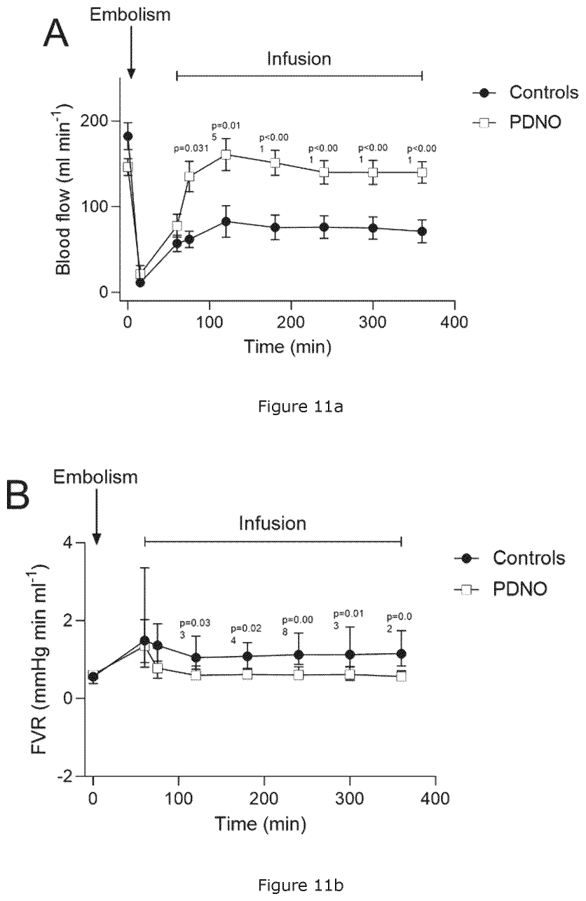

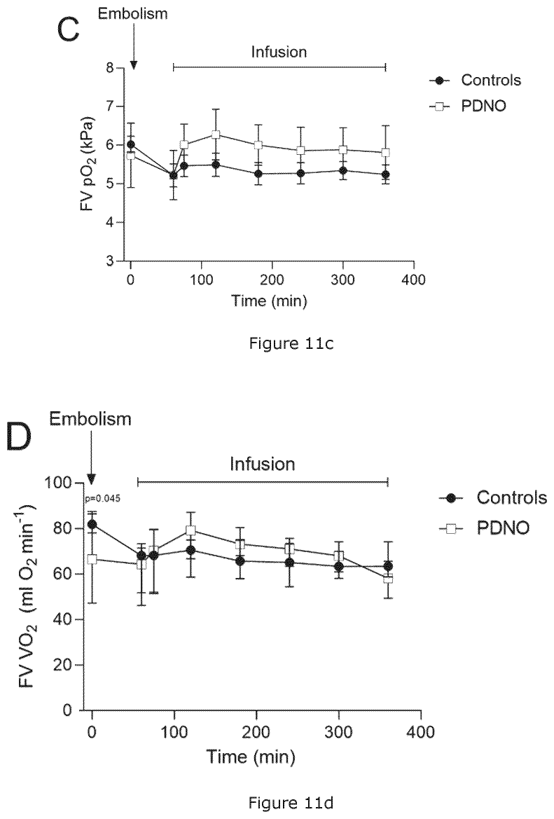

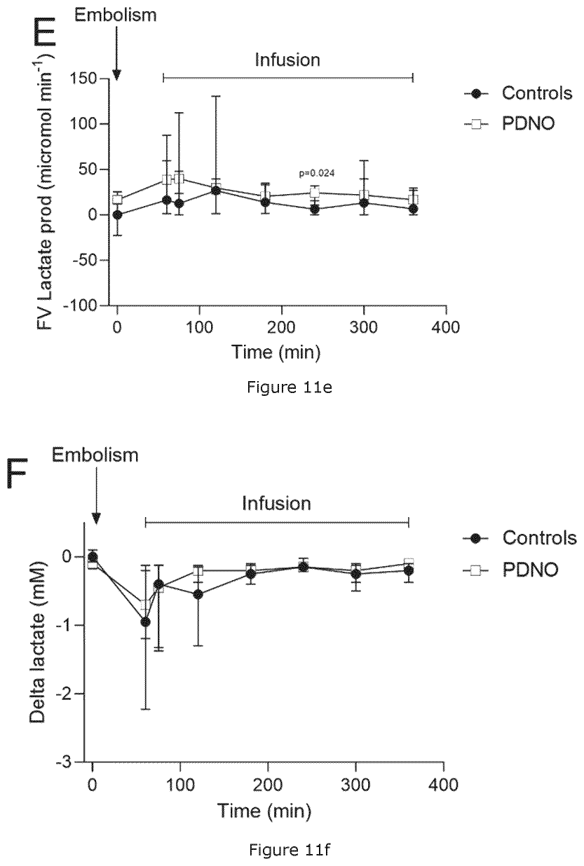

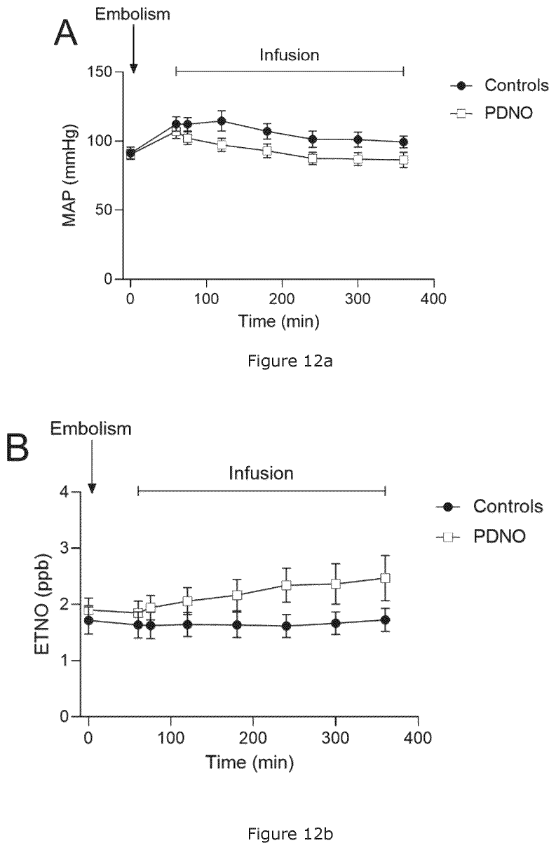

The PDNO group and the control group were similar at baseline except for cardiac output (CO) and femoral venous oxygen consumption (FV O₂) that were higher in the control group at baseline. Blood flow in the common femoral artery (CFA) was extinguished 15 minutes after embolisation in both groups and significantly higher in the PDNO group after start of infusion (FIG. 11). Likewise, femoral venous resistance (FVR) was significantly lower in the PDNO after one hour of infusion (FIG. 12). Heart rate, CO and MAP were similar between the groups.

Femoral venous (FV) pO₂, FV VO2 and FV lactate production were all higher in the PDNO group compared to the control group, although not significant (FIG. 11). Femoral venous CO₂ level was significant higher in the control group at three hours of infusion and FV O₂ levels were generally higher in the PDNO group although not significant. Likewise, ETNO and MHb levels were higher, and pO₂ generally lower, in the PDNO group although not significant (FIG. 12).

### Conclusions

The data show that femoral arterial infusion in porcine experimental acute limb embolization increase femoral blood flow compared to controls with no systemic side effects. Further analyses will show if this vasodilating effect also translates into improved metabolism and less injury of the lower limb.

1. A compound of formula (I): 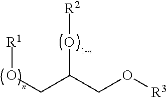
wherein R¹, R² and R³ each independently represent H or —NO,
wherein n is 0 or 1;
wherein when n is 0, R¹ is H; and
wherein when n is 1, R² is H,
provided that at least one of R¹, R² and R³ represents —NO,
for use in the treatment of a condition, wherein the compound of formula (I) is administered to a patient in need thereof via intra-arterial infusion in a dose of from about 0.01 to 3000 nmol kg⁻¹ min⁻¹.

2. The compound for use according to claim 1, wherein the compound of formula (I) is administered at a dose of from about 0.01 to about 300 nmol kg⁻¹ min⁻¹, for example from about 1 to about 300 nmol kg⁻¹ min⁻¹, such as from about 1 to about 100 nmol kg⁻¹ min¹, or from about 10 to about 3000 nmol kg⁻¹ min⁻¹.

3. The compound for use according to claim 1, wherein the compound of formula (I) is administered at a dose of from about 1 to about 30 nmol kg⁻¹ min⁻¹.

4. The compound for use according to claim 1, wherein the compound of formula (I) is administered continuously, optionally for a time period of up to 14 days, such as 7 days.

5. The compound for use according to claim 1, wherein the compound of formula (I) is administered via intra-arterial infusion to the femoral artery and/or the carotid artery.

6. The compound for use according to any one of claim 5, wherein the compound of formula (I) is administered via intra-arterial infusion to the femoral artery and/or the carotid artery at a dose of from about 1 to about 300 nmol kg⁻¹ min⁻¹, such as 1 to about 60 nmol kg⁻¹ min⁻¹, for example 1 to about 50 nmol kg⁻¹ min⁻¹, such as 1 to about 30 nmol kg⁻¹ min⁻¹.

7. The compound for use according to claim 1, wherein the compound of formula (I) is administered via intra-arterial infusion to the superior mesenteric artery.

8. The compound for use according to claim 7, wherein the compound of formula (I) is administered via intra-arterial infusion to the superior mesenteric artery at a dose of from about 3 to about 3000 nmol kg⁻¹ min⁻¹, such as from about 30 to about 1000 nmol kg⁻¹ min⁻¹, for example from about 100 to about 1000 nmol kg⁻¹ min⁻¹.

9. The compound for use according to claim 1, wherein the compound of formula (I) is administered via intra-arterial infusion to the renal artery.

10. The compound for use according to claim 9, wherein the compound of formula (I) is administered via intra-arterial infusion to the renal artery at a dose of from about 0.01 to about 300 nmol kg⁻¹ min⁻¹, for example from about 1 to about 30 nmol kg⁻¹ min⁻¹.

11. The compound for use according to claim 1, wherein the compound is for use in the treatment of a condition wherein NO has a beneficial effect.

12. The compound for use according to claim 1, wherein the condition is selected from the group consisting of
acute pulmonary vasoconstriction of different genesis; pulmonary hypertension of different genesis, including primary hypertension and secondary hypertension; preeclampsia; eclampsia; conditions of different genesis in need of vasodilation; erectile dysfunction; systemic hypertension of different genesis; regional vasoconstriction of different genesis; local vasoconstriction of different genesis; acute heart failure (with or without preserved ejection fraction (HFpEF)); coronary heart disease; myocardial infarction; ischemic heart disease; angina pectoris; instable angina; cardiac arrhythmia; acute pulmonary hypertension in cardiac surgery patients; acidosis; inflammation of the airways; cystic fibrosis; COPD; immotile cilia syndrome; inflammation of the lung; pulmonary fibrosis; acute lung injury (ALI); adult respiratory distress syndrome; acute pulmonary oedema; acute mountain sickness; asthma; bronchitis; hypoxia of different genesis; ischemic diseases of different genesis; stroke; cerebral vasoconstriction; inflammation of the gastrointestinal tract; gastrointestinal dysfunction; gastrointestinal complication; IBD; Crohn's disease; ulcerous colitis; liver disease; pancreas disease; inflammation of the bladder of the urethral tract; inflammation of the urinary bladder and ureters of the urethral tract; inflammation of the skin; diabetic ulcers; diabetic neuropathy; psoriasis; inflammation of different genesis; wound healing; organ protection in ischemia-reperfusion conditions; organ transplantation; tissue transplantation; cell transplantation; acute kidney disease; uterus relaxation; cervix relaxation; thromboembolic diseases, including diseases that are complicated by thromboembolism, such as various blood diseases; arterial occlusion, such as arterial embolism; and conditions where smooth muscle relaxation is needed.

13. The compound for use according to claim 12, wherein the condition to be treated is selected from the group consisting of ischemic diseases of different genesis; thromboembolic diseases including diseases that are complicated by thromboembolism, such as various blood diseases; arterial thrombosis; peripheral ischemia (limb ischemia); thromboembolic stroke; pulmonary embolism; acute mesenteric ischemia (mesenteric arterial occlusion); acute renal artery occlusion; arterial stenosis; arterial occlusion; infarction of the spleen; infarction of the liver, infarction of the lung; Kawasaki disease; and arterial embolism.

14. A compound of formula (I): 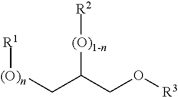
wherein R¹, R² and R³ each independently represent H or —NO,
wherein n is 0 or 1;
wherein when n is 0, R¹ is H; and
wherein when n is 1, R² is H,
provided that at least one of R¹, R² and R³ represents —NO,
for use in the treatment of thromboembolic disease.

15. The compound for use according to claim 14, wherein the compound is administered via intravenous and/or intraarterial infusion.

16. The compound for use according to claim 14, wherein the compound is administered at a dose of from about 0.01 to 3000 nmol kg⁻¹ min⁻¹.

17. The compound for use according to claim 14, wherein the compound is administered at a dose of from about 0.01 to about 300 nmol kg⁻¹ min⁻¹, for example from about 1 to about 300 nmol kg⁻¹ min⁻¹.

18. The compound for use according to claim 17 wherein the compound is administered at a dose of from about 1 to about 300 nmol kg⁻¹ min⁻¹.

19. The compound for use according to claim 17 wherein the compound is administered at a dose of from about 1 to about 10 nmol kg⁻¹ min⁻¹.

20. The compound for use according to claim 14, wherein the compound is administered via intra-arterial infusion to the femoral artery, the carotid arterial, the renal artery or the superior mesenteric artery.

21. (canceled)
